(19) United States
(12) Patent Application Publication
(10) Pub. No.: US 2012/0329661 A1
(43) Pub. Date: Dec. 27, 2012

(54) Isotope Labeling-Assisted Quantification (iLAQ) of Biological Compounds

(76) Inventor: ZengKui Guo, Rochester, MN (US)

(21) Appl. No.: 13/472,103

(22) Filed: May 15, 2012

Related U.S. Application Data
(60) Provisional application No. 61/487,526, filed on May 18, 2011.

(51) Int. Cl. C40B 20/08; C07C 229/46; C07D 295/13; C07C 211/35; C40B 40/04; C40B 50/08

(52) U.S. Cl. 506/6; 506/15; 506/27; 544/402; 564/455; 562/507

(57) ABSTRACT

The present invention relates to a plurality of isotopically labeled compounds (“tag isotopomers”) and individual labeled compounds, which are useful for labeling samples of analytes, such as biological compounds. The present invention further relates to methods of labeling and quantifying analytes using these tag isotopomers.

This application claims the benefit of priority of U.S. Prov. Appl. No. 61/487,526, filed May 18, 2011, which is incorporated herein by reference in its entirety.

### TECHNICAL FIELD

The present invention relates to a plurality of isotopically labeled compounds (“tag isotopomers”) and individual labeled compounds, which are useful for labeling samples of analytes, such as biological compounds. The present invention further relates to methods of labeling and quantifying analytes using these tag isotopomers.

### BACKGROUND

The current methods for the quantification of biological compounds, such as glucose, amino acids, fatty acids, proteins, and peptides in the clinical and research setting can be inefficient, complex, and costly. For example, quantification of biological compounds by mass spectrometry may require several repeated analyses in order to obtain accurate and precise results. Accordingly, there is a need to develop analytical methods and compounds for use in those methods which will increase efficiency. The present invention addresses this need and others.

### SUMMARY

The present invention provides a plurality of isotopically labeled compounds (“tag isotopomers”), which are useful for labeling samples of analytes. In general, each of the tag isotopomers in the plurality have the same structural formula but differ as to their isotopic compositions. In some embodiments, each of the tag isotopomers in the plurality differ by 1 integer atomic mass unit (e.g., ¹³C versus C, D vs. H, or ¹⁵N vs. N at one position in the structural formula).

The tag isotopomers may be used to label and quantify analytes such as biological compounds (e.g., amino acids, proteins, fatty acids, or carbohydrates) that have either a free carboxyl group, aldehyde group, or amine group (e.g., a primary amine or secondary amine group, preferably a primary amine group). Each of the tag isotopomers in a group may be utilized to derivatize one sample of analyte. The samples are then mixed, and the biological compounds isolated by means such as chromatography and analyzed by a mass spectrometry method such as tandem mass spectrometry (MS/MS) in a single run. For example, a plurality of 16 compounds may be used to determine the concentration of a protein from 16 different samples of analyte simultaneously, thereby increasing analytical productivity and reducing analytical errors.

Accordingly, the present invention provides, inter alia, a plurality of compounds of Formula I:

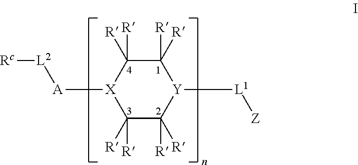

or salts thereof; comprising a non-isobaric series of compounds; wherein:

each compound in the series has the same structural formula but a different isotopic substitution pattern;

n is an integer selected from 0, 1, 2, and 3;

Z is selected from —C(═O)OR¹, —C(═O)X¹, and —NHR²;

X¹ is halogen;

R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy; or

R² is a protecting group;

X is selected from C(R′) and ¹³C(R′); and Y is selected from C(R′) and ¹³C(R′); or

X is selected from N and ¹⁵N; and Y is selected from N and ¹⁵N;

when n is 1, 2, or 3 and X is N or ¹⁵N, then A is absent; or

when (1) X is C(R′) or ¹³C(R′) and n is 1, 2, or 3; or (2) n is 0, then A is N(R^{f}), or ¹⁵N(R^{f});

L¹ is -G¹-G²-G³-G⁴-G⁵-G⁶-; wherein G¹ is attached to Z;

L² is -E¹-E²-E³-E⁴-E⁵-E⁶-E⁷-E⁸; wherein E¹ is attached to A;

R^{f} is —F¹—F²—F³—F⁴—F⁵—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

each R^{c} is independently selected from H and D;

each R^{a}, R^{b}, and R^{b′} is independently selected from H and D;

each R′ is independently selected from H and D; and

C1, C2, C3, and C4 of the ring are independently carbon or carbon-13.

In some embodiments, the plurality is a plurality of compounds of Formula II:

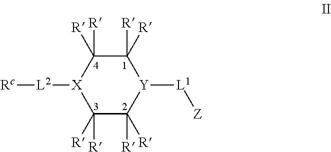

or salts thereof; wherein:

X is selected from N and ¹⁵N;

Y is selected from N and ¹⁵N.

In some embodiments, the plurality is a plurality of compounds of Formula III:

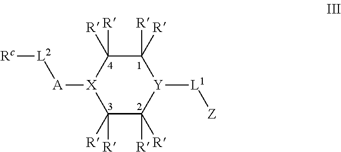

or salts thereof; wherein:

A is —N(R^{f})—, or —¹⁵N(R^{f})—;

X is selected from C(R′) and ¹³C(R′); and

Y is selected from C(R′) and ¹³C(R′).

In some embodiments, the plurality of compounds is plurality of compounds of Formula IV:

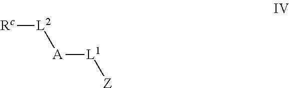

or salts thereof; wherein A is —N(R^{f})—, or —¹⁵N(R^{f})—.

The present invention further provides particular compounds of the plurality. Accordingly, in some embodiments, the present invention provides compounds of Formula Ia:

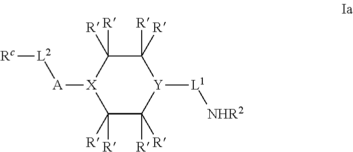

or salts thereof; wherein:

each compound in the plurality has the same structural formula provided that each compound has a different isotopic substitution pattern;

R² is independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy; or

R² is a protecting group;

X is selected from C(R′) and ¹³C(R′); and Y is selected from C(R′) and ¹³C(R′); or

X is selected from N and ¹⁵N; and Y is selected from N and ¹⁵N;

when X is N or ¹⁵N, then A is absent; or

when X is C(R′) or ¹³C(R′), then A is N(R^{f}) or ¹⁵N(R^{f});

L¹ is -G¹-G²-G³-G⁴-G⁵-G⁶-; wherein G¹ is attached to Z;

L² is -E¹-E²-E³-E⁴-E⁵-E⁶-E⁷-E⁸; wherein E¹ is attached to A;

R^{f} is —F¹—F²—F³—F⁴—F⁵—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

each R^{c} is independently selected from H and D;

each R^{a}, R^{b}, and R^{b′} is independently selected from H and D;

each R′ is independently selected from H and D; and

C1, C2, C3, and C4 of the ring are independently carbon or carbon-13;

provided that the compound contains at least one atom selected from ¹³C, ¹⁵N, or D.

The present invention further provides compounds of Formula V:

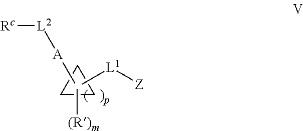

or salts thereof; wherein:

Z is selected from —C(═O)OR¹, —C(═O)X¹, and —NHR²;

A is —N(R^{f})— or —¹⁵N(R^{f})—;

L¹ is -G¹-G²-G³-G⁴-G⁵-G⁶-; wherein G¹ is attached to Z;

L² is -E¹-E²-E³-E⁴-E⁵-E⁶-E⁷-E⁸-; wherein E¹ is attached to A;

R^{f} is —F¹—F²—F³—F⁴—F⁵—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

p is a integer selected from 1, 2, 3, 4, and 5;

m is integer equal to 2+(2*p);

the carbon atoms of the ring in Formula V are each independently carbon or carbon-13;

each R^{c} is independently selected from H and D;

each R^{a}, R^{b}, and R^{b′} is independently selected from H and D;

each R′ is independently selected from H and D;

X¹ is halogen; and

R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₉ aryl, C₆₋₁₉ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy; or

R² is a protecting group;

provided that the compound contains at least one atom selected from ¹³C, ¹⁵N, or D.

In some embodiments, compounds of Formula V have Formula Va:

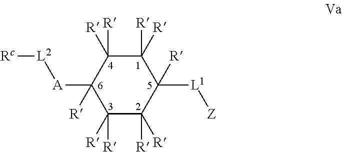

or salt thereof; wherein:

Z is selected from —C(═O)OR¹, —C(═O)X¹, and —NHR²;

A is —N(R^{f})— or —¹⁵N(R^{f})—;

L¹ is -G¹-G²-G³-G⁴-G⁵-G⁶-; wherein G¹ is attached to Z;

L² is -E¹-E²-E³-E⁴-E⁵-E⁶-E⁷-E⁸; wherein E¹ is attached to A;

R^{f} is —F¹—F²—F³—F⁴—F⁵—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

each R^{c} is independently selected from H and D;

each R^{a}, R^{b}, and R^{b′} is independently selected from H and D;

each R′ is independently selected from H and D;

X¹ is halogen;

R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy; or

R² is a protecting group; and

C1, C2, C3, C4, C5, and C6 of the ring in Formula Va are independently carbon or carbon-13;

provided that the compound contains at least one atom selected from ¹³C, ¹⁵N, or D.

In some embodiments, the present invention provides a plurality of compounds of Formula VI:

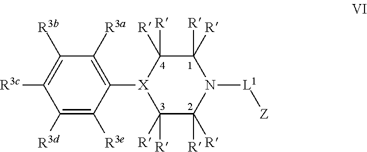

or salts thereof; comprising a non-isobaric series of compounds.

In some embodiments, the present invention provides a compound of Formula VI: or salt thereof.

The present invention also provides kits comprising a plurality of compounds selected from the plurality of compounds described herein.

The present invention further provides methods of forming a plurality of labeled analyte samples, comprising reacting independently each of q individual samples of the analyte with a different compound selected from the plurality of compounds described herein, to form a plurality of q labeled samples; wherein:

q is equal to the total number of compounds in the plurality of compounds;

each individual sample of the analyte is reacted with a different compound in the plurality of compounds; and

each of the q labeled samples comprises a labeled analyte.

The present invention further provides methods of quantifying an analyte, comprising:

mixing together the plurality of q labeled samples prepared as described supra, or any embodiment thereof; and

isolating the labeled analytes; and

quantifying the analyte by conducting an mass spectrometry analysis on the labeled analytes obtained from the isolating;

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts a plurality of compounds having a 1,4-piperazine core (compounds of Formula II, wherein Z is —NH₂). Stars indicate either a ¹⁵N or ¹³C is present as indicated. D atoms are indicated as D.

FIG. 2 depicts a plurality of compounds having a 1,4-piperazine core (compounds of Formula II, wherein Z is —CO₂H). Stars indicate either a ¹⁵N or ¹³C is present as indicated. D atoms are indicated as D.

FIG. 3 depicts a plurality of compounds having a 1,4-piperazine core (compounds of Formula II, wherein Z is —NH₂). Stars indicate either a ¹⁵N or ¹³C is present as indicated. D atoms are indicated as D.

FIG. 4 depicts a plurality of compounds having a 1,4-piperazine core (compounds of Formula II, wherein Z is —CO₂H). Stars indicate either a ¹⁵N or ¹³C is present as indicated. D atoms are indicated as D.

FIG. 5 depicts a plurality of compounds having a 1,4-piperazine core (compounds of Formula II, wherein Z is —NH₂). Stars indicate either a ¹⁵N or ¹³C is present as indicated. D atoms are indicated as D.

FIG. 6 depicts a plurality of compounds having a 1,4-piperazine core (compounds of Formula II, wherein Z is —CO₂H). Stars indicate either a ¹⁵N or ¹³C is present as indicated. D atoms are indicated as D.

FIG. 7 depicts a plurality of compounds having a 1,4-piperazine core (compounds of Formula II, wherein Z is —NH₂). Stars indicate either a ¹⁵N or ¹³C is present as indicated. D atoms are indicated as D.

FIG. 8 depicts a plurality of compounds having a 1,4-piperazine core (compounds of Formula II, wherein Z is —CO₂H). Stars indicate either a ¹⁵N or ¹³C is present as indicated. D atoms are indicated as D.

FIG. 9 depicts a plurality of compounds having a 1,4-piperazine core (compounds of Formula II, wherein Z is —NH₂). Stars indicate either a ¹⁵N or ¹³C is present as indicated. D atoms are indicated as D.

FIG. 10 depicts a plurality of compounds having a 1,4-piperazine core (compounds of Formula II, wherein Z is —CO₂H). Stars indicate either a ¹⁵N or ¹³C is present as indicated. D atoms are indicated as D.

FIG. 11 depicts a plurality of compounds having a 1,4-piperazine core (compounds of Formula II, wherein Z is —NH₂, nitrogen atoms not shown). Stars indicate either a ¹⁵N or ¹³C is present as indicated. D atoms are indicated as D.

FIG. 12 depicts a plurality of compounds having a 1,4-piperazine core (compounds of Formula II, wherein Z is —CO₂H). Stars indicate either a ¹⁵N or ¹³C is present as indicated. D atoms are indicated as D.

FIG. 13 depicts a plurality of compounds having a cyclohexyl core (compounds of Formula III). Stars indicate either a ¹⁵N or ¹³C is present as indicated. D atoms are indicated as D.

FIG. 14 depicts a plurality of compounds having a cyclohexyl core (compounds of Formula III). Stars indicate either a ¹⁵N or ¹³C is present as indicated. D atoms are indicated as D.

FIG. 15 depicts a plurality of compounds having a trialkylamine core (compounds of Formula IV). Stars indicate either a ¹⁵N or ¹³C is present as indicated. D atoms are indicated as D.

FIG. 16 depicts a plurality of compounds having a trialkylamine core (compounds of Formula IV).

FIG. 17 depicts a proton ion mass spectrum of a combination of d0-glucose and d0-PPEA.

FIG. 18 depicts a proton ion mass spectrum of a combination of d0-glucose and d4-PPEA.

FIG. 19 depicts a proton ion mass spectrum of a combination of d2-glucose and d0-PPEA.

FIG. 20 depicts a proton ion mass spectrum of a combination of d2-glucose and d4-PPEA.

Like reference symbols in the various drawings indicate like elements.

### DETAILED DESCRIPTION

The present invention provides, inter alia, a plurality of compounds of Formula I:

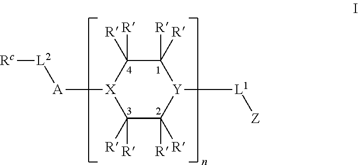

or salts thereof; comprising a non-isobaric series of compounds; wherein:

each compound in the series has the same structural formula but a different isotopic substitution pattern;

n is an integer selected from 0, 1, 2, or 3;

Z is selected from —C(═O)OR¹, —C(═O)X¹, and —NHR²;

X¹ is halogen;

R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy;

R² is a protecting group;

X is selected from C(R′) and ¹³C(R′); and Y is selected from C(R′) and ¹³C(R′); or

X is selected from N and ¹⁵N; and Y is selected from N and ¹⁵N;

when n is 1, 2, or 3 and X is N or ¹⁵N, then A is absent; or

when (1) X is C(R′) or ¹³C(R′) and n is 1, 2, or 3; or (2) n is 0, then A is N(R^{f}), or ¹⁵N(R^{f});

L¹ is -G¹-G²-G³-G⁴-G⁵-G⁶-; wherein G¹ is attached to Z;

L² is -E¹-E²-E³-E⁴-E⁵-E⁶-E⁷-E⁸; wherein E¹ is attached to A;

R^{f} is —F¹—F²—F³—F⁴—F⁵—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

each R^{c} is independently selected from H and D;

each R^{a}, R^{b}, and R^{b′} is independently selected from H and D;

each R′ is independently selected from H and D; and

C1, C2, C3, and C4 of the ring are independently carbon or carbon-13.

As used herein, the term “non-isobaric series of compounds” means that each compound in the plurality has the same structural formula provided that each compound in the series has a different isotopic substitution pattern. A non-limiting example of a non-isobaric series of compounds would include three piperazinyl-methylamine compounds, one compound without any isotopic substitution; one compound wherein one carbon atom is replaced by ¹³C; and one compound wherein three carbon atoms are replaced by ¹³C.

In some embodiments, the plurality comprises at least 2, at least 3, at least 4, at least 5, at least 6, at least 7, at least 8, at least 9, at least 10, at least 11, at least 12, at least at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, or at least 85 compounds. In some embodiments, the plurality comprises at least 16 compounds. In some embodiments, the plurality comprises at least 20 compounds. In some embodiments, the plurality comprises at least 22 compounds. In some embodiments, the plurality comprises at least 23 compounds. In some embodiments, the plurality comprises at least 36 compounds. In some embodiments, the plurality is selected from a non-isobaric series of 16 compounds. In some embodiments, the plurality is selected from a non-isobaric series of 20 compounds. In some embodiments, the plurality is selected from a non-isobaric series of 22 compounds. In some embodiments, the plurality is selected from a non-isobaric series of 23 compounds.

In some embodiments, the plurality is selected from a non-isobaric series of 36 compounds. Each of these embodiments may also apply, as appropriate, to the plurality and kits described infra.

In some embodiments:

each compound has a formula weight which is 1 integer atomic mass unit higher than the previous compound in the plurality; and

the compound with the highest formula weight in the plurality is isotopically labeled with at least one of each of ¹³C, ¹⁵N, and D.

In some embodiments, the isotopic substitution in said plurality is D only. In some embodiments, the isotopic substitution in said plurality is ¹³C only. In some embodiments, the isotopic substitution in said plurality is ¹⁵N only.

In some embodiments:

each compound has a formula weight which is 1 integer atomic mass unit higher than the previous compound in the plurality;

the compound with the lowest formula weight in the plurality is not isotopically labeled with ¹³C, ¹⁵N, or D; and

the compound with the highest formula weight in the plurality is isotopically labeled with at least one of each of ¹³C, ¹⁵N, and D.

In some embodiments, R¹ and R² are each H.

In some embodiments, Z is —C(═O)OH. In some embodiments, Z is —NH₂.

In some embodiments, n is 0 or 1.

In some embodiments, the plurality is a plurality of compounds of Formula II:

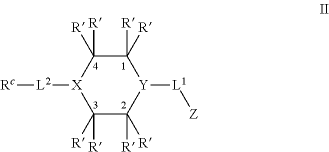

or salts thereof; wherein:

X is selected from N and ¹⁵N;

Y is selected from N and ¹⁵N.

In some embodiments, R¹ and R² are each H.

In some embodiments, Z is —C(═O)OH. In some embodiments, Z is —NH₂.

In some embodiments, L¹ is -G¹-G²-; wherein G¹ is attached to Z.

In some embodiments, L¹ is -G¹-.

In some embodiments, L² is absent.

In some embodiments:

L² is selected from -E¹-, E¹-E²-, -E¹-E²-E³-, -E¹-E²-E³-E⁴-, -E¹-E²-E³-E⁴-E⁵-, and -E¹-E²-E³-E⁴-E⁵-E⁶-; and

E¹, E², E³, E⁴, E⁵, and E⁶ are each independently —C(R^{b})₂— or —¹³C(R^{b})₂—.

In some embodiments:

L² is -E¹-E²-; and

E¹ and E² are each independently —C(R^{b})₂— or —¹³C(R^{b})₂—.

In some embodiments:

L² is -E¹-E²-E³-; and

E¹, E², and E³ are each independently —C(R^{b})₂— or —¹³C(R)₂—.

In some embodiments:

L² is -E¹-E²-E³-E⁴-E⁵-E⁶-; and

E¹, E², E³, E⁴, E⁵, and E⁶ are each independently —C(R^{b})₂— or —¹³C(R^{b})₂—.

In some embodiments, for the compound with the highest formula weight in the plurality:

C1, C2, C3, and C4 of the ring are each carbon-13;

X is ¹⁵N;

Y is ¹⁵N;

G¹ is —¹³C(R^{a})₂—; and

each R′ is D.

In some embodiments, for the compound with the highest formula weight in the plurality:

C1, C2, C3, and C4 of the ring are each carbon-13;

X is ¹⁵N;

Y is ¹⁵N;

G¹ is —¹³C(R^{a})₂—;

G² is —C(R^{a})₂—;

R^{a} is D;

R^{c} is D; and

each R′ is D.

In some embodiments, for the compound with the highest formula weight in the plurality:

C1, C2, C3, and C4 of the ring are each carbon-13;

X is ¹⁵N;

Y is ¹⁵N;

G¹ is —¹³CD₂-;

E² and E³ are each —¹³CH₂—;

E¹ is —¹³CD₂-; and

each R′ is D.

In some embodiments, for the compound with the highest formula weight in the plurality:

C1, C2, C3, and C4 of the ring are each carbon-13;

X is ¹⁵N;

Y is ¹⁵N;

G¹ is —¹³CD₂-;

E² is —¹³CH₂—;

E¹ is —¹³CD₂-; and

each R′ is D.

In some embodiments, for the compound with the highest formula weight in the plurality:

C1, C2, C3, and C4 of the ring are each carbon-13;

X is ¹⁵N;

Y is ¹⁵N;

G¹ is —¹³CD₂-;

each R′ is D; and

E¹, E², E³, E⁴, E⁵, and E⁶ are each —¹³CD₂-.

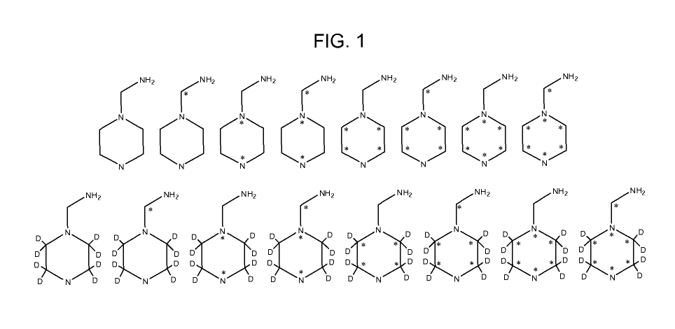

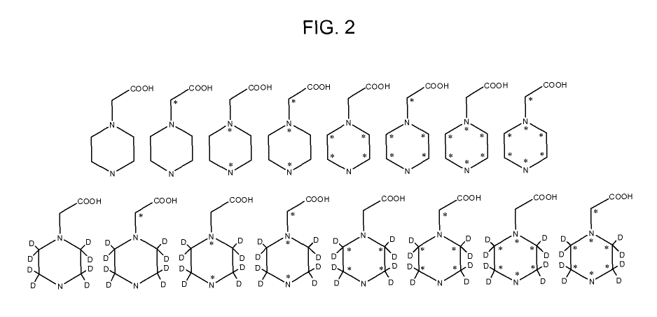

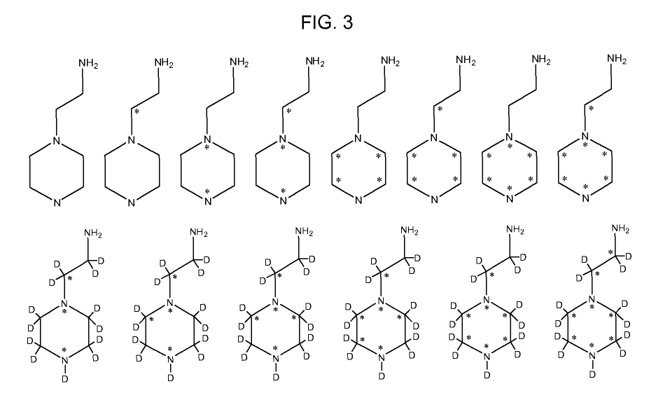

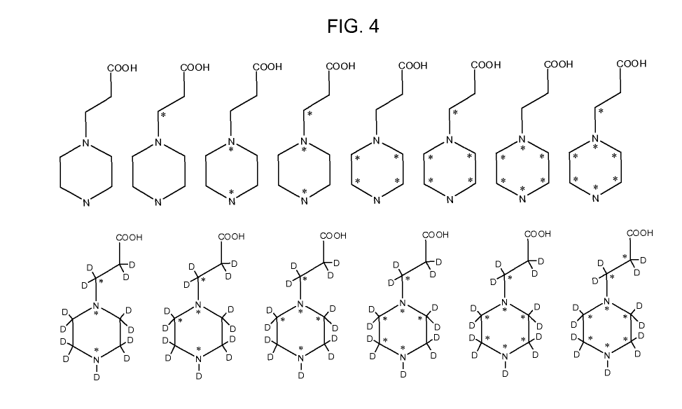

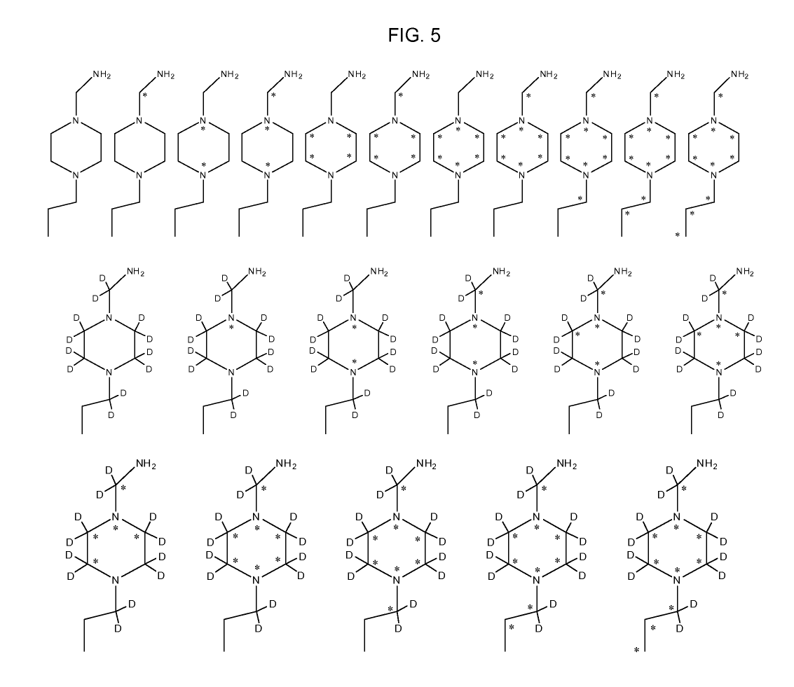

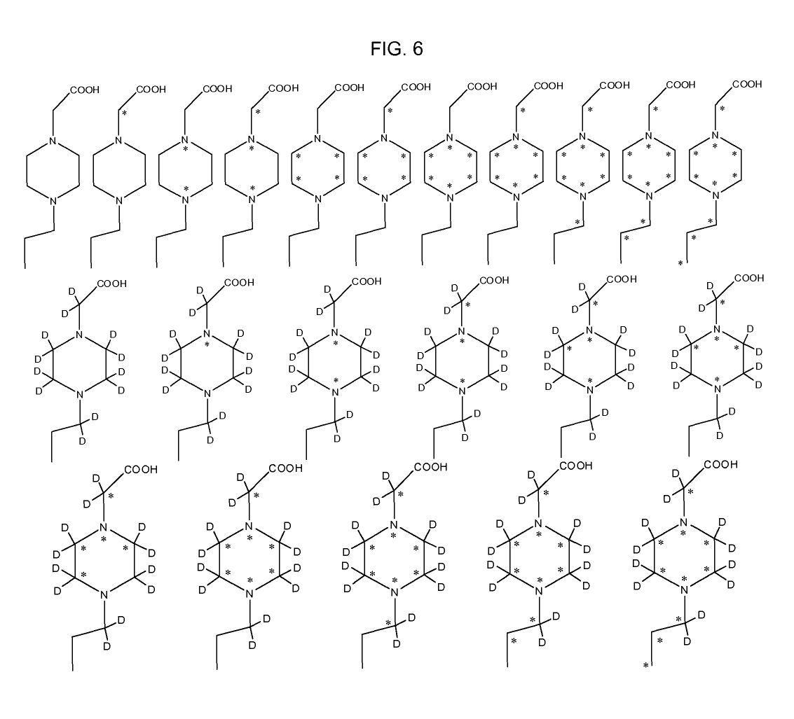

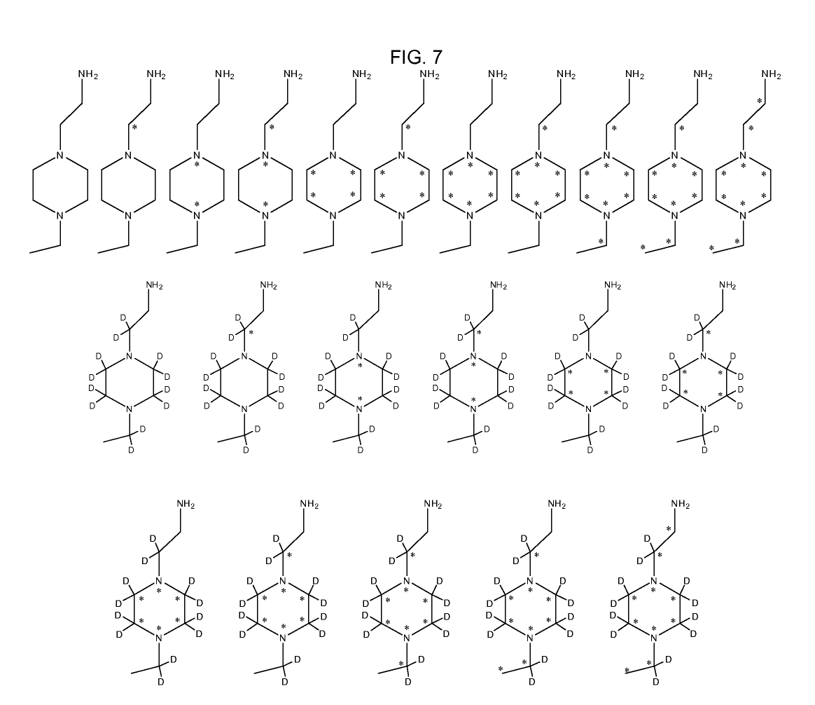

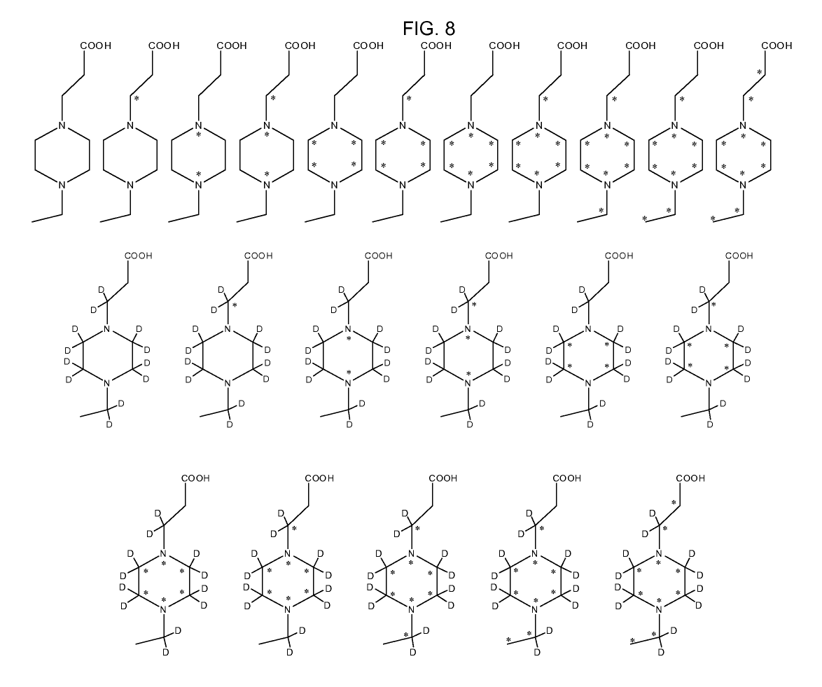

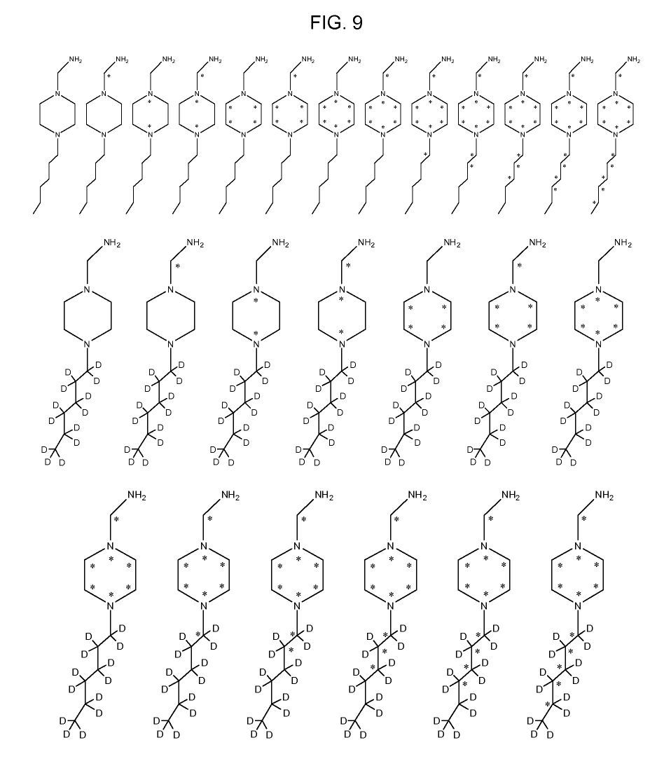

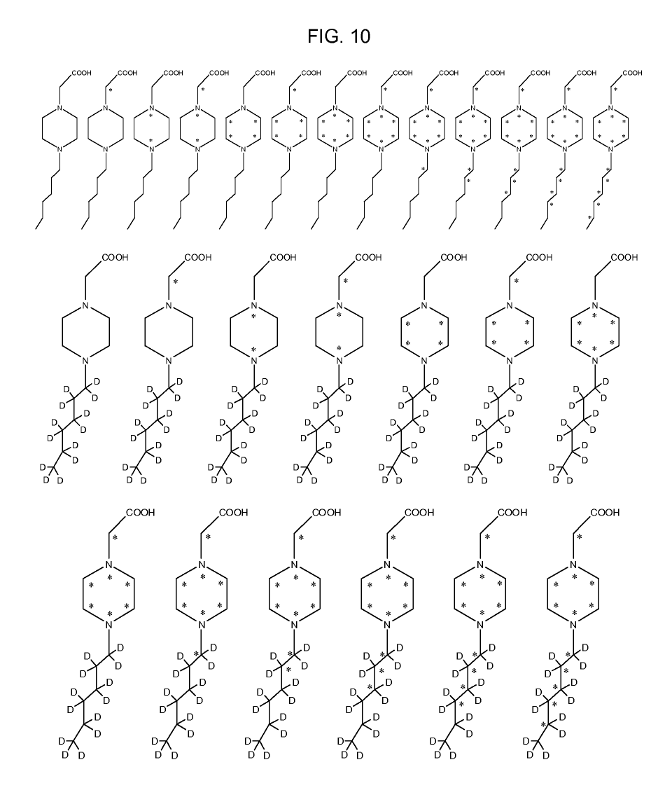

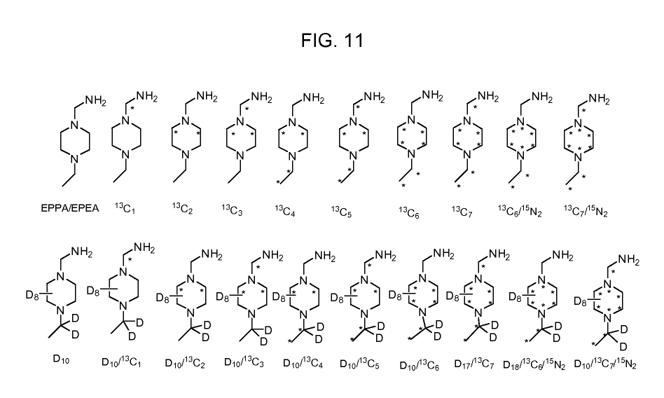

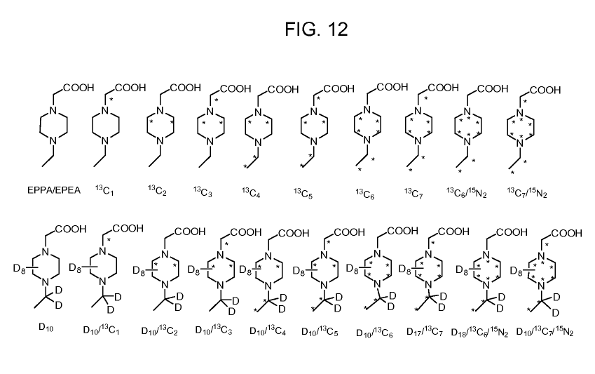

In some embodiments, the plurality comprises the compounds in any one of FIGS. 1 to 12. In some embodiments, the plurality is selected from the compounds in any one of FIGS. 1 to 12. In some embodiments, the plurality comprises at least 8 compounds. In some embodiments, the plurality comprises at least 16 compounds. In some embodiments, the plurality comprises at least 22 compounds. In some embodiments, the plurality comprises at least 36 compounds.

In some embodiments, the plurality is a plurality of compounds of Formula III:

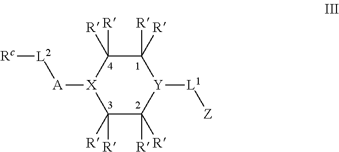

or salts thereof; wherein:

A is —N(R^{f})—, or —¹⁵N(R^{f})—;

X is selected from C(R′) and ¹³C(R′); and

Y is selected from C(R′) and ¹³C(R′).

In some embodiments, R¹ and R² are each H.

In some embodiments, Z is —C(═O)OH. In some embodiments, Z is —NH₂.

In some embodiments, L¹ is -G¹-G²-; wherein G¹ is attached to Z. In some embodiments, L¹ is -G¹-.

In some embodiments:

L² is -E¹-;

R^{f} is —F¹—R^{c};

E¹ is —C(R^{b})₂— or —¹³C(R)₂—; and

F¹ is —C(R^{b′})₂— or —¹³C(R^{b′})₂—.

In some embodiments, for the compound with the highest formula weight in the plurality:

C1, C2, C3, and C4 are each carbon-13;

X is ¹³C(R′);

Y is ¹³C(R′);

A is —¹⁵N(R^{f})—;

E¹ is —¹³CH₂—;

F¹ is —¹³CH₂—; and

G¹ is —¹³CD₂-.

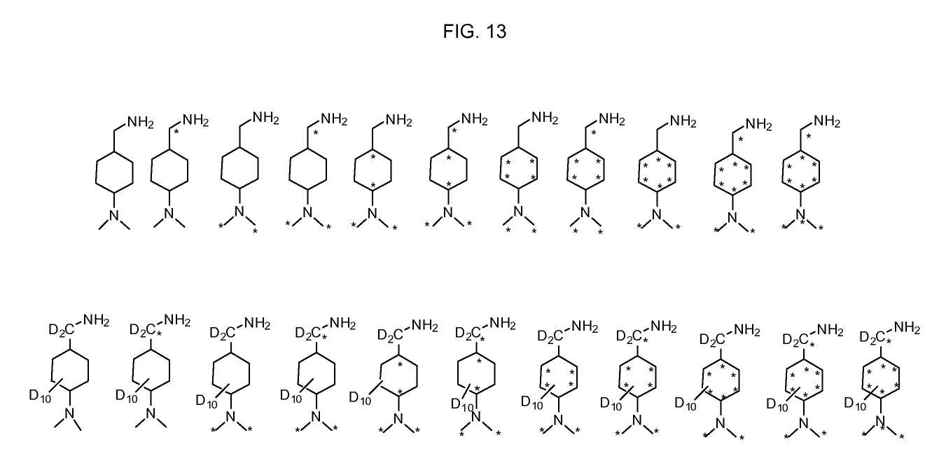

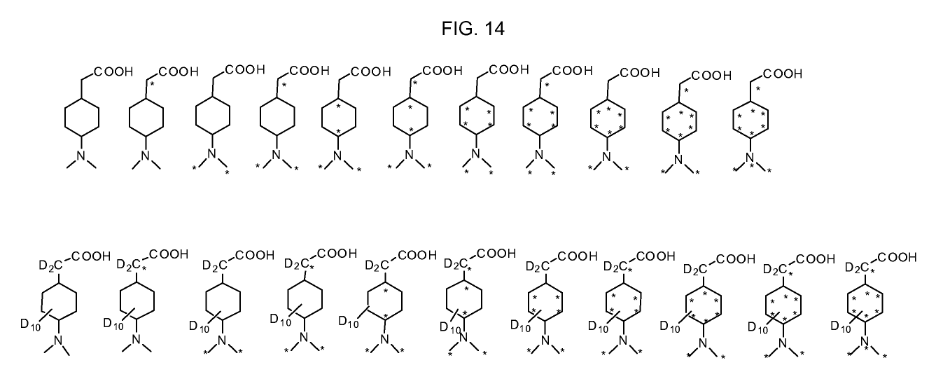

In some embodiments, the plurality comprises the compounds in any one of FIGS. 13 to 14. In some embodiments, the plurality is selected from the compounds in any one of FIGS. 13 to 14. In some embodiments, the plurality comprises at least 8 compounds. In some embodiments, the plurality comprises at least 16 compounds. In some embodiments, the plurality comprises at least 20 compounds. In some embodiments, the plurality comprises at least 22 compounds.

In some embodiments, the plurality of compounds is plurality of compounds of Formula IV:

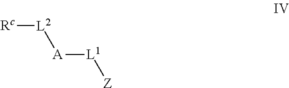

or salts thereof; wherein A is —N(R^{f})—, or —¹⁵N(R^{f})—.

In some embodiments, L¹ is -G¹-G²-.

In some embodiments:

L² is selected from -E¹-E²-E³-; wherein E¹ is attached to A;

R^{f} is selected from —F¹—F²—F³—R^{c};

E¹, E², and E³ are each independently —C(R^{b})₂— or —¹³C(R^{b})₂—; and

F¹, F², and F³ are each independently absent, —C(R^{b′})₂— or —¹³C(R^{b′})₂—.

In some embodiments, R¹ and R² are each H.

In some embodiments, Z is —C(═O)OH. In some embodiments, Z is —NH₂.

In some embodiments, for the compound with the highest formula weight in the plurality:

A is —¹⁵N(R^{f})—;

E¹, E², and E³ are each —¹³CD₂-;

F¹, F², and F³ are each —¹³CD₂-; and

G¹ and G² are each —¹³CH₂—.

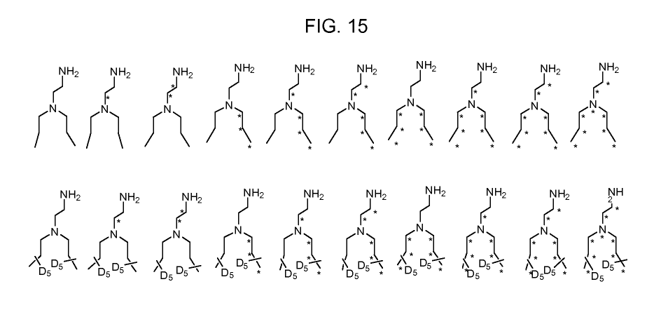

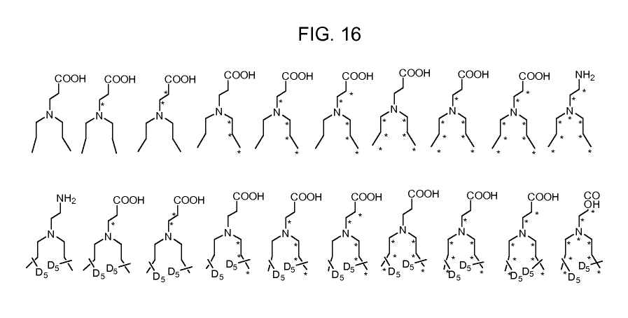

In some embodiments, the plurality comprises the compounds in any one of FIGS. 15 to 16. In some embodiments, the plurality is selected from the compounds in any one of FIGS. 15 to 16. In some embodiments, the plurality comprises at least 8 compounds. In some embodiments, the plurality comprises at least 16 compounds. In some embodiments, the plurality comprises at least 20 compounds. In some embodiments, the plurality comprises at least 22 compounds.

In some embodiments, the plurality comprises compounds of Formula VI:

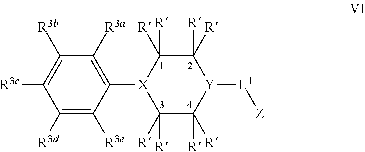

or salts thereof; comprising a non-isobaric series of compounds; wherein:

each compound in the series has the same structural formula but a different isotopic substitution pattern;

Z is selected from —C(═O)OR¹, —C(═O)X¹, and —NHR²;

X¹ is halogen;

R¹ and R² are each independently selected from H and C₁₋₆ alkyl;

X is selected from C(R′), ¹³C(R′), N and ¹⁵N;

R^{3a}, R^{3b}, R^{3c}, R^{3d}, and R^{3e} are independently selected from H, D, and C₁₋₄ alkyl;

L¹ is -G¹-G²-G³-G⁴-G⁵-G⁶-; wherein G¹ is attached to Z;

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

R^{a} is selected from H and D; and

C1, C2, C3, and C4 of the ring are independently carbon or carbon-13.

In some embodiments:

R^{3a}, R^{3b}, R^{3c}, R^{3d}, and R^{3e} are independently selected from H and D;

L¹ is -G¹-G²-;

G¹ and G² are each independently —C(R^{a})₂— or —¹³C(R^{a})₂—;

Z is —C(═O)OH or —NH₂; and

X is selected from C(R′) and ¹³C(R′).

In some embodiments, for at least some of compounds of the plurality, R^{a} is D. In some embodiments, for at least some of compounds of the plurality, R^{a} is D and the remaining variables are not isotopically substituted.

In some embodiments, R^{3a}, R^{3b}, R^{3c}, R^{3d}, and R^{3e} are independently selected from H and D.

In some embodiments, L¹ is -G¹-G²-.

In some embodiments, G¹ and G² are each independently —C(R^{a})₂— or —¹³C(R^{a})₂—.

In some embodiments, Z is —C(═O)OH or —NH₂.

In some embodiments, X is selected from C(R′) and ¹³C(R′).

In some embodiments, the present invention provides a compound of Formula VI, or a salt thereof, as defined above, provided that the compound contains at least one atom selected from ¹³C, ¹⁵N, or D.

The following embodiments may apply to the previous embodiments for the compounds of Formulas I, II, III, and IV.

In some embodiments, L¹ is -G¹-G²-; wherein G¹ is attached to Z. In some embodiments, L¹ is -G¹-.

In some embodiments, one compound is not labeled with carbon-13, nitrogen-15 or D.

In some embodiments, the plurality comprises at least one compound, wherein G¹ is —¹³C(R^{a})₂—.

In some embodiments, the plurality comprises at least one compound labeled with carbon-13, nitrogen-15 or both as in any of the preceding embodiments, wherein:

X is ¹⁵N; and Y is ¹⁵N; or

X is ¹³C(R′); and Y is ¹³C(R′).

In some embodiments, the plurality comprises at least one compound labeled with carbon-13, nitrogen-15 or both as in any of the preceding embodiments, wherein A is —¹⁵NR^{f}—.

In some embodiments, the plurality comprises at least one compound labeled with carbon-13, nitrogen-15 or both as in any of the preceding embodiments, wherein G¹ and G² are each —¹³C(R^{b})₂—.

In some embodiments, the plurality comprises at least one compound labeled with carbon-13, nitrogen-15 or both as in any of the preceding embodiments, wherein G′, G², and G³ are each —¹³C(R^{b})₂—.

In some embodiments, the plurality comprises at least one compound labeled with carbon-13, nitrogen-15 or both as in any of the preceding embodiments, wherein G¹, G², G³, and G⁴ are each —¹³C(R^{b})₂—.

In some embodiments, the plurality comprises at least one compound labeled with carbon-13, nitrogen-15 or both as in any of the preceding embodiments, wherein G¹, G², G³, G⁴, and G⁵ are each —¹³C(R^{b})₂—.

In some embodiments, the plurality comprises at least one compound labeled with carbon-13, nitrogen-15 or both as in any of the preceding embodiments, wherein G′, G², G³, G⁴, G⁵, and G⁶ are each —¹³C(R^{b})₂—.

In some embodiments, the plurality comprises at least one compound labeled with carbon-13, nitrogen-15 or both as in any of the preceding embodiments, wherein C1 is carbon-13.

In some embodiments, the plurality comprises at least one compound labeled with carbon-13, nitrogen-15 or both as in any of the preceding embodiments, wherein C2 is carbon-13.

In some embodiments, the plurality comprises at least one compound labeled with carbon-13, nitrogen-15 or both as in any of the preceding embodiments, wherein C3 is carbon-13.

In some embodiments, the plurality comprises at least one compound labeled with carbon-13, nitrogen-15 or both as in any of the preceding embodiments, wherein C4 is carbon-13.

In some embodiments, the plurality comprises at least one compound labeled with carbon-13, nitrogen-15 or both as in any of the preceding embodiments, wherein C1 and

C2 are each carbon-13.

In some embodiments, the plurality comprises at least one compound labeled with carbon-13, nitrogen-15 or both as in any of the preceding embodiments, wherein C1, C2, and C3 are each carbon-13.

In some embodiments, the plurality comprises at least one compound labeled with carbon-13, nitrogen-15 or both as in any of the preceding embodiments, wherein C1, C2, C3, and C4 are each carbon-13.

In some embodiments, the plurality comprises at least one compound labeled with carbon-13, nitrogen-15 or both as in any of the preceding embodiments, wherein E¹ is —¹³C(R_{b})₂—.

In some embodiments, the plurality comprises at least one compound labeled with carbon-13, nitrogen-15 or both as in any of the preceding embodiments, wherein E¹ and E² are each —¹³C(R_{b})₂—.

In some embodiments, the plurality comprises at least one compound labeled with carbon-13, nitrogen-15 or both as in any of the preceding embodiments, wherein E¹, E², and E³ are each —¹³C(R_{b})₂—.

In some embodiments, the plurality comprises at least one compound labeled with carbon-13, nitrogen-15 or both as in any of the preceding embodiments, wherein E¹, E², E³, and E⁴ are each —¹³C(R_{b})₂—.

In some embodiments, the plurality comprises at least one compound labeled with carbon-13, nitrogen-15 or both as in any of the preceding embodiments, wherein E¹, E², E³, E⁴, and E⁵ are each —¹³C(R_{b})₂—.

In some embodiments, the plurality comprises at least one compound labeled with carbon-13, nitrogen-15 or both as in any of the preceding embodiments, wherein E¹, E², E³, E⁴, E⁵, and E⁶ are each —¹³C(R_{b})₂—.

In some embodiments, the plurality comprises at least one compound labeled with carbon-13, nitrogen-15 or both as in any of the preceding embodiments, wherein E¹, E², E³, E⁴, E⁵, E⁶, and E⁷ are each —¹³C(R_{b})₂—.

In some embodiments, the plurality comprises at least one compound labeled with carbon-13, nitrogen-15 or both as in any of the preceding embodiments, wherein E¹, E², E³, E⁴, E⁵, E⁶, E⁷, and E⁸ are each —¹³C(R_{b})₂—.

In some embodiments, the plurality comprises at least one compound labeled with carbon-13, nitrogen-15 or both as in any of the preceding embodiments, wherein F¹ is —¹³C(R_{b′})₂—

In some embodiments, the plurality comprises at least one compound labeled with carbon-13, nitrogen-15 or both as in any of the preceding embodiments, wherein F¹ and F² are each —¹³C(R^{b′})₂—.

In some embodiments, the plurality comprises at least one compound labeled with carbon-13, nitrogen-15 or both as in any of the preceding embodiments, wherein F¹, F², and F³ are each —¹³C(R^{b′})₂—.

In some embodiments, the plurality comprises at least one compound labeled with carbon-13, nitrogen-15 or both as in any of the preceding embodiments, wherein F¹, F², F³, and F⁴ are each —¹³C(R^{b′})₂—.

In some embodiments, the plurality comprises at least one compound labeled with carbon-13, nitrogen-15 or both as in any of the preceding embodiments, wherein F¹, F², F³, F⁴, and F⁵ are each —¹³C(R^{b′})₂—.

In some embodiments, the plurality comprises at least one compound labeled with carbon-13, nitrogen-15 or both as in any of the preceding embodiments, wherein F¹, F², F³, F⁴, F⁵, and F⁶ are each —¹³C(R^{b′})₂—.

In some embodiments, the plurality comprises at least one compound labeled with carbon-13, nitrogen-15 or both as in any of the preceding embodiments, wherein F¹, F², F³, F⁴, F⁵, F⁶, and F⁷ are each —¹³C(R^{b′})₂—.

In some embodiments, the plurality comprises at least one compound labeled with carbon-13, nitrogen-15 or both as in any of the preceding embodiments, wherein F¹, F², F³, F⁴, F⁵, F⁶, F⁷, and F⁸ are each —¹³C(R^{b′})₂—.

In some embodiments, the plurality comprises at least one compound labeled with carbon-13, nitrogen-15 or both as in any of the preceding embodiments and further having w D atoms wherein w is 1+(the total number of atoms labeled with carbon-13 or nitrogen-15 in the highest formula weight compound of the plurality which is not labeled with D).

In some embodiments, the plurality comprises at least one compound labeled with carbon-13, nitrogen-15 or both as in any of the preceding embodiments, wherein each R′ is D.

In some embodiments, the plurality comprises at least one compound labeled with carbon-13, nitrogen-15 or both as in any of the preceding embodiments, wherein G¹ is perdeuterated.

In some embodiments, the plurality comprises at least one compound labeled with carbon-13, nitrogen-15, deuterium, or combination thereof as in any of the preceding embodiments, wherein each R^{a} is D.

In some embodiments, the plurality comprises at least one compound labeled with carbon-13, nitrogen-15, deuterium, or combination thereof as in any of the preceding embodiments, wherein each R^{b} is D.

In some embodiments, the plurality comprises at least one compound labeled with carbon-13, nitrogen-15, deuterium, or combination thereof as in any of the preceding embodiments, wherein each R^{b′} is D.

The present invention further provides a kit comprising a plurality of compounds selected from the plurality of compounds described supra, or any embodiment thereof.

In some embodiments, the kit further comprises instructions for reacting the plurality of compounds with an analyte having one or more amino or carboxyl groups. In some embodiments, the kit further comprises instructions for use of the plurality of compounds in any one of the methods described herein, or embodiment thereof.

The present invention further provides a method of forming a plurality of labeled analyte samples, comprising reacting independently each of q individual samples of the analyte with a different compound selected from the plurality of compounds described supra, or any embodiment thereof, to form a plurality of q labeled samples; wherein:

q is equal to the total number of compounds in the plurality of compounds;

each individual sample of the analyte is reacted with a different compound in the plurality of compounds; and

each of the q labeled samples comprises a labeled analyte.

In some embodiments, the analyte is a carboxylic acid or amine containing compound. Carboxylic acids and amines are major classes of natural compounds that exist in many domains including plants, animals, human body, natural materials, etc. Both classes can be quantified using the plurality or kits of compounds described herein.

For example, fatty acids, amino acids, keto acids, and di- and tricarboxylic acids are common acid species with significant clinical implications as various human diseases affect their metabolism and thus concentration in blood or tissues. Therefore, clinical measurement of these acids is often required in order to help doctor diagnose diseases. These acids are also common compounds in research laboratories and chemical industry settings and their quantitation is a routine. Appropriate carboxylic acid analytes, which can be quantified by use of the plurality or kits wherein Z is selected from Z is —NH₂ or —NHR², include, but are not limited to,

Fatty acids, including short chain, medium chain and long chain; saturated and unsaturated (monosaturated and polyunsaturated);

Amino acids;

Keto acids (e.g. pyruvic acid);

Aromatic carboxylic acids (e.g. benzoic acid);

Dicarboxylic acids (e.g. succinic acid);

Tricarboxylic acids (e.g. citric acid);

Hydroxy carboxylic acids (e.g. lactate, glycolic acid);

Heterocyclic acids (e.g. nicotinic acid); and

Other compounds that contain one or more —COOH groups (carboxyl group).

Other analytes include amine compounds. Amines are important metabolites and signaling molecules in human and animal bodies. Amino acids are essential nutrients. Many other amines are important industrial materials and compounds for research. Their clinical quantification is important for diagnosis. Many amines are used as medications such as antidepressants. Appropriate amine analytes, which can be quantified by using the kit or plurality wherein Z is —C(═O)OR¹ or —C(═O)X¹, include, but are not limited to:

Amino acids (various types e.g. branched chain, cyclic, sulfur, basic, acidic, etc);

Amino sugars (e.g. glucosamine);

Aromatic amines (e.g. aniline);

Neurotransmitters (e.g. serotonin, norepinephrine, dopamine, histamine, etc);

Drugs (amphetamine, dobutamine, imipramine, etc);

Monoamines, Polyamines; and

Other compounds that contain one or more —NH₂ group (primary amine).

In some embodiments, the analyte has at least one amine group and Z is —C(═O)OH. In some embodiments, the analyte has at least one carboxyl group and Z is —NH₂. In some embodiments, the reacting is performed in the presence of a coupling agent.

In some embodiments, the analyte is selected from a protein, a peptide, an amino acid, and a fatty acid. In some embodiments, the analyte is a protein.

The present invention also provides a method of quantifying an analyte, comprising:

mixing together the plurality of q labeled samples prepared as described supra, or any embodiment thereof; and

isolating the labeled analytes; and

quantifying the analyte by conducting an mass spectrometry analysis on the labeled analytes obtained from the isolating;

In some embodiments, the mass spectrometry analysis comprises use of tandem mass spectrometry.

In some embodiments, the isolating comprises chromatography. In some embodiments, the isolating is high performance liquid chromatography.

The present invention further provides a compound of Formula Ia:

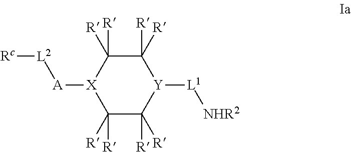

or salt thereof; wherein:

each compound in the plurality has the same structural formula provided that each compound has a different isotopic substitution pattern;

R² is independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy;

R² is a protecting group;

X is selected from C(R′) and ¹³C(R′); and Y is selected from C(R′) and ¹³C(R′); or

X is selected from N and ¹⁵N; and Y is selected from N and ¹⁵N;

when X is N or ¹⁵N, then A is absent; or

when X is C(R′) or ¹³C(R′), then A is N(R^{f}), or ¹⁵N(R^{f});

L¹ is -G¹-G²-G³-G⁴-G⁵-G⁶-; wherein G¹ is attached to Z;

L² is -E¹-E²-E³-E⁴-E⁵-E⁶-E⁷-E⁸-; wherein E¹ is attached to A;

R^{f} is —F¹—F²—F³—F⁴—F⁵—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

each R^{c} is independently selected from H and D;

each R^{a}, R^{b}, and R^{b′} is independently selected from H and D;

each R′ is independently selected from H and D; and

C1, C2, C3, and C4 of the ring are independently carbon or carbon-13;

provided that the compound contains at least one atom selected from ¹³C, ¹⁵N, or D.

In some embodiments, R² is H. In some embodiments, the embodiments for n, X, Y, L¹, G¹, G², G³, G⁴, G⁵, G⁶, L², E¹, E², E³, E⁴, E⁵, E⁶, E⁷, E⁸, R^{f}, F¹, F², F³, F⁴, F⁵, F⁶, F⁷, F⁸, R^{c}, R^{a}, R^{b}, R^{b′}, R′, A, C1, C2, C3, and C4 can be any of the embodiments described supra for Formulas I, II, III, IV, and V.

The present invention further provides a compound of Formula V:

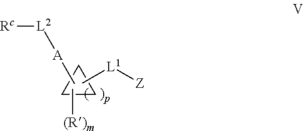

or salt thereof; wherein:

Z is selected from —C(═O)OR¹, —C(═O)X¹, and —NHR²;

A is —N(R^{f})— or —¹⁵N(R^{f})—;

L¹ is -G¹-G²-G³-G⁴-G⁵-G⁶-; wherein G¹ is attached to Z;

L² is -E¹-E²-E³-E⁴-E⁵-E⁶-E⁷-E⁸-; wherein E¹ is attached to A;

R^{f} is —F¹—F²—F³—F⁴—F^{S}—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

p is a integer selected from 1, 2, 3, 4, and 5;

m is integer equal to 2+(2*p);

the carbon atoms of the ring in Formula V are each independently carbon or carbon-13;

each R^{c} is independently selected from H and D;

each R^{a}, R^{b}, and R^{b′} is independently selected from H and D;

each R′ is independently selected from H and D;

X¹ is halogen; and

R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy; or

R² is a protecting group;

provided that the compound contains at least one atom selected from ¹³C, ¹⁵N, or D.

In some embodiments, the embodiments for Z, n, L¹, G¹, G², G³, G⁴, G⁵, G⁶, L², E¹, E², E³, E⁴, E⁵, E⁶, E⁷, E⁸, R^{f}, F¹, F², F³, F⁴, F⁵, F⁶, F⁷, F⁸, R^{c}, R^{a}, R^{b}, R^{b′}, R′, A, C1, C2, C3, and C4 can be any of the embodiments described supra for Formulas I, II, III, IV, and V. In some embodiments, C5 and C6 are —¹³CH—. In some embodiments, p is 4. In some embodiments, m is 10. In some embodiments, R′ is D.

In some embodiments, the compound of Formula V has Formula Va:

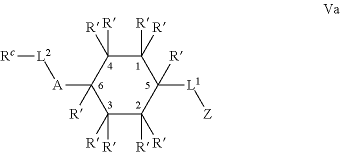

or salt thereof; wherein:

Z is selected from —C(═O)OR¹, —C(═O)X¹, and —NHR²;

A is —N(R^{f})— or —¹⁵N(R^{f})—;

L¹ is -G¹-G²-G³-G⁴-G⁵-G⁶-; wherein G¹ is attached to Z;

L² is -E¹-E²-E³-E⁴-E⁵-E⁶-E⁷-E⁸; wherein E¹ is attached to A;

R^{f} is —F¹—F²—F³—F⁴—F⁵—F⁶—F⁷—F⁸—R^{c};

G¹ is —C(R^{a})₂— or —¹³C(R^{a})₂—;

G², G³, G⁴, G⁵, and G⁶ are each independently absent, —C(R^{a})₂—, or —¹³C(R^{a})₂—;

E¹, E², E³, E⁴, E⁵, E⁶, E⁷, and E⁸ are each independently absent, —C(R^{b})₂—, or —¹³C(R^{b})₂—;

F¹, F², F³, F⁴, F⁵, F⁶, F⁷, and F⁸ are each independently absent, —C(R^{b′})₂—, or —¹³C(R^{b′})₂—;

each R^{c} is independently selected from H and D;

each R^{a}, R^{b}, and R^{b′} is independently selected from H and D;

each R′ is independently selected from H and D;

X¹ is halogen;

R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy; or

R² is a protecting group; and

C1, C2, C3, C4, C5, and C6 of the ring in Formula Va are independently carbon or carbon-13;

provided that the compound contains at least one atom selected from ¹³C, ¹⁵N, or D.

In some embodiments, R² is H.

In some embodiments, Z is selected from —C(═O)OH and —NH₂. In some embodiments, Z is —C(═O)OH. In some embodiments, Z is —NH₂.

At various places in the present specification, substituents of compounds are disclosed in groups or in ranges. It is specifically intended that the compounds include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features, which are, for clarity, described in the context of separate embodiments, can also be provided in combination in a single embodiment. Conversely, various features which are, for brevity, described in the context of a single embodiment, can also be provided separately or in any suitable subcombination.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R. In another example, when an optionally multiple substituent is designated in the form:

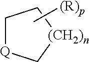

then it is understood that substituent R can occur p number of times on the ring, and R can be a different moiety at each occurrence. It is understood that each R group may replace any hydrogen atom attached to a ring atom, including one or both of the (CH₂)ₙ hydrogen atoms. Further, in the above example, should the variable Q be defined to include hydrogens, such as when Q is the to be CH₂, NH, etc., any floating substituent such as R in the above example, can replace a hydrogen of the Q variable as well as a hydrogen in any other non-variable component of the ring.

The compounds described herein can be asymmetric (e.g., having one or more stereocenters). All stereoisomers, such as enantiomers and diastereomers, are intended unless otherwise indicated. Compounds described herein that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds described herein may be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as (3-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein, or pharmaceutically acceptable salts or N-oxides thereof, further include hydrates and solvates, as well as anhydrous and non-solvated forms. Compounds described herein can also include all isotopes of atoms occurring in the intermediates or final compounds. Isotopes include those atoms having the same atomic number but different mass numbers. For example, isotopes of hydrogen include tritium and deuterium.

The term, “compound” as used herein is meant to include all stereoisomers, tautomers, and isotopes of the structures depicted.

As used herein, the phrase “optionally substituted” means unsubstituted or substituted. As used herein, the term “substituted” means that a hydrogen atom is removed and replaced by a substitutent. It is understood that substitution at a given atom is limited by valency.

As used herein, the term “Cₙ₋ₘ alkyl”, employed alone or in combination with other terms, refers to a saturated hydrocarbon group that may be straight-chain or branched, having n to m carbon atoms. In some embodiments, the alkyl group contains 1 to 12, 1 to 8, 1 to 6, or 1 to 4 carbon atoms. Examples of alkyl moieties include, but are not limited to, chemical groups such as methyl, ethyl, n-propyl, isopropyl, n-butyl, tert-butyl, isobutyl, sec-butyl, 2-methyl-1-butyl, n-pentyl, 3-pentyl, n-hexyl, 1,2,2-trimethylpropyl, n-heptyl, n-octyl, and the like.

As used herein, “Cₙ₋ₘ alkenyl”, employed alone or in combination with other terms, refers to an alkyl group having one or more double carbon-carbon bonds and n to m carbon atoms. In some embodiments, the alkenyl moiety contains 2 to 10 or 2 to 6 carbon atoms. Example alkenyl groups include, but are not limited to, ethenyl, n-propenyl, isopropenyl, n-butenyl, sec-butenyl, and the like.

As used herein, “Cₙ₋ₘ alkynyl”, employed alone or in combination with other terms, refers to an alkyl group having one or more triple carbon-carbon bonds, which may also optionally have one or more double carbon-carbon bonds, and having n to m carbon atoms. In some embodiments, the alkynyl moiety contains 2 to 10 or 2 to 6 carbon atoms. Example alkenyl groups include, but are not limited to, ethenyl, n-propenyl, isopropenyl, n-butenyl, sec-butenyl, and the like.

As used herein, the term “amine”, employed alone or in combination with other terms, refers to a group of formula —NH₂ or —NHR, wherein R is C₁₋₆ alkyl.

As used herein, the term “cyano”, employed alone or in combination with other terms, refers to a group of formula —CN.

As used herein, the terms “halo” and “halogen”, employed alone or in combination with other terms, refer to fluoro, chloro, bromo, and iodo. In some embodiments, halogen is fluoro.

As used herein, the term “Cₙ₋ₘ haloalkyl”, employed alone or in combination with other terms, refers to an alkyl group having from n to m carbon atoms and one halogen atom to 2x+1 halogen atoms which may be the same or different, where “x” is the number of carbon atoms in the alkyl group. In some embodiments, the halogen atoms are fluoro atoms. In some embodiments, the alkyl group has 1 to 6 or 1 to 4 carbon atoms. An example of a haloalkyl group is —CF₃.

As used herein, “Cₙ₋ₘ haloalkoxy”, employed alone or in combination with other terms, refers to a group of formula —O-haloalkyl, wherein the haloalkyl group has n to m carbon atoms. In some embodiments, the alkyl group has 1 to 6 or 1 to 4 carbon atoms. An example haloalkoxy group is —OCF₃.

As used herein, the term “Cₙ₋ₘ fluorinated alkyl”, employed alone or in combination with other terms, refers to a haloalkyl wherein the halogen atoms are selected from fluorine. In some embodiments, fluorinated Cₙ₋ₘ haloalkyl is fluoromethyl, difluoromethyl, or trifluoromethyl.

As used herein, the term “Cₙ₋ₘ cycloalkyl”, employed alone or in combination with other terms, refers to a non-aromatic cyclic hydrocarbon moiety, which may optionally contain one or more alkenylene groups as part of the ring structure, and which has n to m ring member carbon atoms. Cycloalkyl groups can include mono- or polycyclic (e.g., having 2, 3, or 4 fused, bridged, or Spiro rings) ring systems. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. The term “cycloalkyl” also includes bridgehead cycloalkyl groups and spirocycloalkyl groups. As used herein, “bridgehead cycloalkyl groups” refers to non-aromatic cyclic hydrocarbon moieties containing at least one bridgehead carbon, such as admantan-1-yl. As used herein, “spirocycloalkyl groups” refers to non-aromatic hydrocarbon moieties containing at least two rings fused at a single carbon atom, such as spiro[2.5]octane and the like. In some embodiments, the cycloalkyl group has 3 to 14 ring members, 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. One or more ring-forming carbon atoms of a cycloalkyl group can be oxidized to form carbonyl linkages. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, the cycloalkyl group is admanatan-1-yl.

As used herein, the term “Cₙ₋ₘ cycloalkyl-Cₒ₋ₚ alkyl”, employed alone or in combination with other terms, refers to a group of formula -alkylene-cycloalkyl, wherein the cycloalkyl portion has n to m carbon atoms and the alkylene portion has o to p carbon atoms. In some embodiments, the alkylene portion has 1 to 4, 1 to 3, 1 to 2, or 1 carbon atom(s). In some embodiments, the alkylene portion is methylene. In some embodiments, the cycloalkyl portion has 3 to 14 ring members, 3 to 10 ring members, or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group.

As used herein, the term “Cₙ₋ₘ heterocycloalkyl”, “Cₙ₋ₘ heterocycloalkyl ring”, or “Cₙ₋ₘ heterocycloalkyl group”, employed alone or in combination with other terms, refers to non-aromatic ring or ring system, which may optionally contain one or more alkenylene or alkynylene groups as part of the ring structure, which has at least one heteroatom ring member independently selected from nitrogen, sulfur and oxygen, and which has n to m ring member carbon atoms. Heterocycloalkyl groups can include mono- or polycyclic (e.g., having 2, 3 or 4 fused, bridged, or Spiro rings) ring systems. In some embodiments, the heterocycloalkyl group is a monocyclic or bicyclic group having 1, 2, 3, or 4 hetereoatoms independently selected from nitrogen, sulfur and oxygen. Also included in the definition of heterocycloalkyl are moieties that have one or more aromatic rings fused (i.e., having a bond in common with) to the non-aromatic ring, for example, 1,2,3,4-tetrahydro-quinoline and the like. Heterocycloalkyl groups can also include bridgehead heterocycloalkyl groups and spiroheterocycloalkyl groups. As used herein, “bridgehead heterocycloalkyl group” refers to a heterocycloalkyl moiety containing at least one bridgehead atom, such as azaadmantan-1-yl and the like. As used herein, “spiroheterocycloalkyl group” refers to a heterocycloalkyl moiety containing at least two rings fused at a single atom, such as [1,4-dioxa-8-aza-spiro[4.5]decan-N-yl] and the like. In some embodiments, the heterocycloalkyl group has 3 to 20 ring-forming atoms, 3 to 10 ring-forming atoms, or about 3 to 8 ring forming atoms. The carbon atoms or hetereoatoms in the ring(s) of the heterocycloalkyl group can be oxidized to form a carbonyl, or sulfonyl group (or other oxidized linkage) or a nitrogen atom can be quaternized. In some embodiments, the heterocycloalkyl portion is a C₂₋₇ monocyclic heterocycloalkyl group.

As used herein, the term “Cₙ₋ₘ heterocycloalkyl-Cₒ₋ₚ alkyl”, employed alone or in combination with other terms, refers to a group of formula -alkylene-heterocycloalkyl, wherein the heterocycloalkyl portion has n to m carbon atoms and the alkylene portion has o to p carbon atoms. In some embodiments, the alkylene portion has 1 to 4, 1 to 3, 1 to 2, or 1 carbon atom(s). In some embodiments, the alkylene portion is methylene. In some embodiments, the heterocycloalkyl portion has 3 to 14 ring members, 3 to 10 ring members, or 3 to 7 ring members. In some embodiments, the heterocycloalkyl group is monocyclic or bicyclic. In some embodiments, the heterocycloalkyl portion is monocyclic. In some embodiments, the heterocycloalkyl portion is a C₂₋₇ monocyclic heterocycloalkyl group.

As used herein, the term “Cₙ₋ₘ aryl”, employed alone or in combination with other terms, refers to a monocyclic or polycyclic (e.g., having 2, 3 or 4 fused rings) aromatic hydrocarbon moiety having n to m ring member carbon atoms, such as, but not limited to, phenyl, 1-naphthyl, 2-naphthyl, anthracenyl, phenanthrenyl, and the like. In some embodiments, aryl groups have from 6 to 14 carbon atoms, about 6 to 10 carbon atoms, or about 6 carbons atoms. In some embodiments, the aryl group is a monocyclic or bicyclic group.

As used herein, the term “Cₙ₋ₘ aryl-Cₒ₋ₚ-alkyl”, employed alone or in combination with other terms, refers to a group of formula -alkylene-aryl, wherein the aryl portion has n to m ring member carbon atoms and the alkylene portion has o to p carbon atoms. In some embodiments, the alkylene portion has 1 to 4, 1 to 3, 1 to 2, or 1 carbon atom(s). In some embodiments, the alkylene portion is methylene. In some embodiments, the aryl portion is phenyl. In some embodiments, the aryl group is a monocyclic or bicyclic group. In some embodiments, the arylalkyl group is benzyl.

As used herein, the term “Cₙ₋ₘ heteroaryl”, “Cₙ₋ₘ heteroaryl ring”, or “Cₙ₋ₘ heteroaryl group”, employed alone or in combination with other terms, refers to a monocyclic or polycyclic (e.g., having 2, 3 or 4 fused rings) aromatic hydrocarbon moiety, having one or more heteroatom ring members independently selected from nitrogen, sulfur and oxygen and having n to m ring member carbon atoms. In some embodiments, the heteroaryl group is a monocyclic or bicyclic group having 1, 2, 3, or 4 hetereoatoms independently selected from nitrogen, sulfur and oxygen. Example heteroaryl groups include, but are not limited to, pyrrolyl, azolyl, oxazolyl, thiazolyl, imidazolyl, furyl, thienyl, quinolinyl, isoquinolinyl, indolyl, benzothienyl, benzofuranyl, benzisoxazolyl, imidazo[1,2-b]thiazolyl or the like. The carbon atoms or hetereoatoms in the ring(s) of the heteroaryl group can be oxidized to form a carbonyl, or sulfonyl group (or other oxidized linkage) or a nitrogen atom can be quaternized, provided the aromatic nature of the ring is preserved. In some embodiments, the heteroaryl group has 5 to 10 carbon atoms.

As used herein, the term “Cₙ₋ₘ heteroaryl-Cₒ₋ₚ-alkyl”, employed alone or in combination with other terms, refers to a group of formula -alkylene-heteroaryl, wherein the heteroaryl portion has n to m ring member carbon atoms and the alkylene portion has to p carbon atoms. In some embodiments, the alkylene portion has 1 to 4, 1 to 3, 1 to 2, or 1 carbon atom(s). In some embodiments, the alkylene portion is methylene. In some embodiments, the heteroaryl portion is a monocyclic or bicyclic group having 1, 2, 3, or 4 hetereoatoms independently selected from nitrogen, sulfur and oxygen. In some embodiments, the heteroaryl portion has 5 to 10 carbon atoms.

The term “protecting group” with respect to R² includes the protecting groups for amines described in Greene, et al., Protective Groups in Organic Synthesis, 4d. Ed., Wiley & Sons, 2007, which is incorporated herein by reference in its entirety.

Unless otherwise indicated herein, the point of attachment of a substituent is generally in the last portion of the name (e.g., arylalkyl is attached through the alkylene portion of the group).

The compounds can also include salt forms of the compounds described herein. Examples of salts (or salt forms) include, but are not limited to, mineral or organic acid salts of basic residues such as amines, alkali or organic salts of acidic residues such as carboxylic acids, and the like. Generally, the salt forms can be prepared by reacting the free base or acid with stoichiometric amounts or with an excess of the desired salt-forming inorganic or organic acid or base in a suitable solvent or various combinations of solvents.

The compounds also include pharmaceutically acceptable salts of the compounds disclosed herein. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient. Pharmaceutically acceptable salts, including mono- and bi-salts, include, but are not limited to, those derived from organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties.

The compounds described herein can be prepared in a variety of ways known to one skilled in the art of organic synthesis. The compounds can be synthesized using the methods as hereinafter described below, together with synthetic methods known in the art of synthetic organic chemistry or variations thereon as appreciated by those skilled in the art.

The compounds can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds of the invention.

The processes described herein can be monitored according to any suitable method known in the art. For example, product formation can be monitored by spectroscopic means, such as nuclear magnetic resonance spectroscopy (e.g., ¹H or ¹³C NMR) infrared spectroscopy, spectrophotometry (e.g., UV-visible), or mass spectrometry, or by chromatography such as high performance liquid chromatography (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007, which is incorporated herein by reference in its entirety. Adjustments to the protecting groups and formation and cleavage methods described herein may be adjusted as necessary in light of the various substituents.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Suitable solvents can include halogenated solvents such as carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-dichloroethane, 2-chloropropane, α,α,α-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable ether solvents include: dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, mixtures thereof and the like.

Suitable protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol.

Suitable aprotic solvents can include, by way of example and without limitation, tetrahydrofuran (THF), N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

Suitable hydrocarbon solvents include benzene, cyclohexane, pentane, hexane, toluene, cycloheptane, methylcyclohexane, heptane, ethylbenzene, m-, o-, or p-xylene, octane, indane, nonane, or naphthalene.

Supercritical carbon dioxide and ionic liquids can also be used as solvents.

The reactions of the processes described herein can be carried out at appropriate temperatures which can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures). “Elevated temperature” refers to temperatures above room temperature (about 22° C.).

The reactions of the processes described herein can be carried out in air or under an inert atmosphere. Typically, reactions containing reagents or products that are substantially reactive with air can be carried out using air-sensitive synthetic techniques that are well known to the skilled artisan.

In some embodiments, preparation of compounds can involve the addition of acids or bases to effect, for example, catalysis of a desired reaction or formation of salt forms such as acid addition salts.

Example acids can be inorganic or organic acids. Inorganic acids include hydrochloric acid, hydrobromic acid, sulfuric acid, phosphoric acid, and nitric acid. Organic acids include formic acid, acetic acid, propionic acid, butanoic acid, benzoic acid, 4-nitrobenzoic acid, methanesulfonic acid, p-toluenesulfonic acid, benzenesulfonic acid, tartaric acid, trifluoroacetic acid, propiolic acid, butyric acid, 2-butynoic acid, vinyl acetic acid, pentanoic acid, hexanoic acid, heptanoic acid, octanoic acid, nonanoic acid and decanoic acid.

Example bases include lithium hydroxide, sodium hydroxide, potassium hydroxide, lithium carbonate, sodium carbonate, and potassium carbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include sodium and potassium salts of methyl, ethyl, n-propyl, i-propyl, n-butyl, t-butyl, trimethylsilyl and cyclohexyl substituted amides.

Upon carrying out preparation of compounds according to the processes described herein, the usual isolation and purification operations such as concentration, filtration, extraction, solid-phase extraction, recrystallization, chromatography, and the like may be used, to isolate the desired products.

In some embodiments, the compounds can be substantially isolated. By “substantially isolated” is meant that the compound is at least partially or substantially separated from the environment in which it was formed or detected. Partial separation can include, for example, a composition enriched in the compound or intermediate, or salt thereof. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

As used herein, the expressions, “ambient temperature” and “room temperature,” as used herein, are understood in the art, and refer generally to a temperature, e.g. a reaction temperature, that is about the temperature of the room in which the reaction is carried out, for example, a temperature from about 20° C. to about 30° C.

As used herein, the term “reacting” is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

### EXAMPLES

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Labeled Plurality of 2-(4-Phenylpiperazin-1-Yl)Ethanamine (PPEA) Compounds

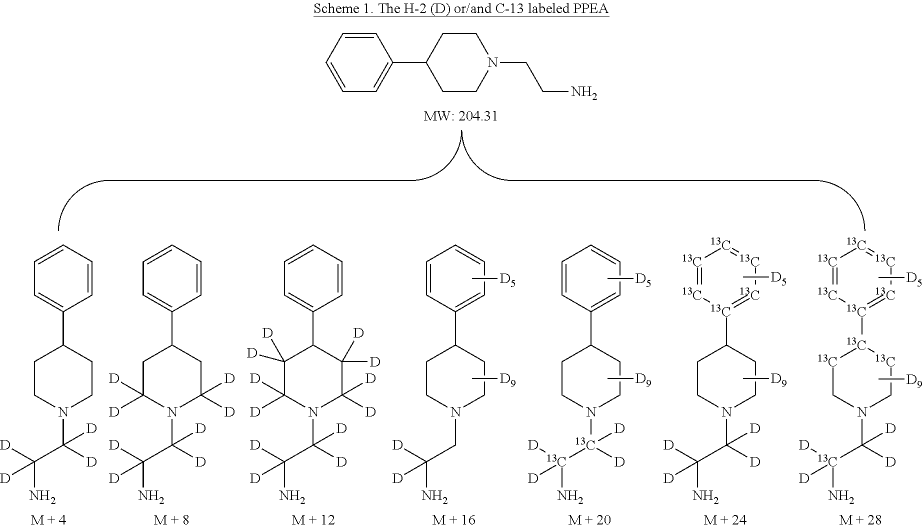

Eight compounds including seven stable isotope (C-13 and H-2) labeled analogs of PPEA are designed as shown in Scheme 1. The molecular weights of the PPEA compounds are increased from M₀ (204) to M₂₈ (232). The synthetic approach to the labeled compounds is based the synthesis shown in Scheme 2. The H-2 and C-13 sources are D₂O, LiAlD₄, [¹³C]CO₂, [¹³C]KCN or [¹³C]NaCN or D₂ gas, which are economically viable starting materials.

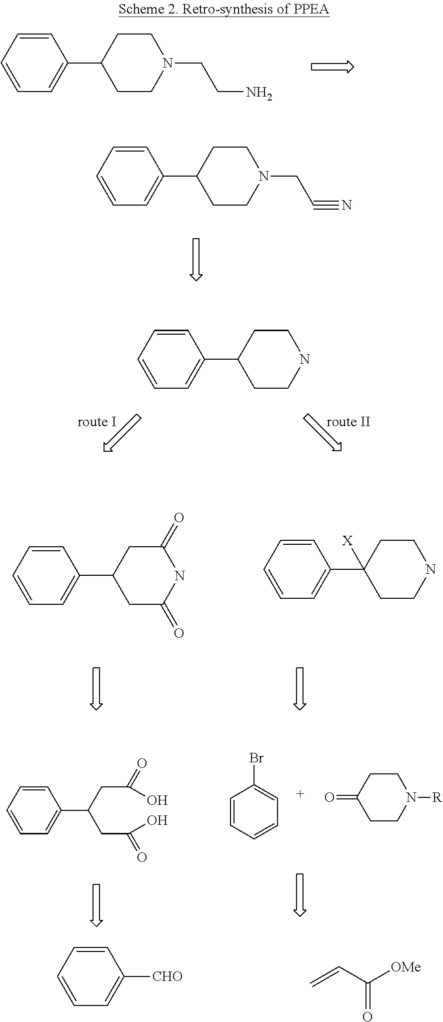

The key new labeling technology is direct H-D exchange reactions with deuterium water (D₂O) catalyzed by Pd—Pt—NaBH₄ or a base under microwave condition. The reduction of carbonyl group with LiAlD₄ is also a simple way to introduce the deuterium into the targets.

There are two major synthetic approaches towards the desired isotope labeled PPEA. The route I is based on an double Aldol-condensation, de-carboxylation and amination to build the piperidine ring. The route II is based on the Michael addition and C—C cross-coupling reactions to generate the phenylpiperidine system.

### Example 2

### Use of Labeled Tags for Relative Quantification of Metabolites

One of the most commonly used procedures in measuring the concentration of an analyte in a medium such as plasma is to add an internal standard to the sample at the beginning of the extraction. Every sample has the same amount of internal standard added. A calibration curve is also prepared where known amounts of analyte are aliquoted together with the same amount of internal standard. The premise is that any losses during the extraction procedure are experienced by both the analyte and the internal standard and the ratio of the two remains constant. It is important that the internal standard has similar chemical properties to the analyte so that it will behave in a similar manner throughout the chemical extraction procedure. For this reason compounds containing stable isotope labeled species such as ¹³C or deuterium make ideal internal standards. While this methodology works well, it has one disadvantage in that only one sample at a time can be analyzed. In many situations the concentration of an analyte (or analytes) in two or more groups of samples (e.g. a control and diseased state) are being compared, but each sample can only be run individually. The use of chemical tags, each with a different chemical property, such as a different number of stable isotope labels, can be added to the samples each to form a derivative with a different molecular weight. After preparation of the derivative the samples from the different groups are mixed together and then analysed in the same run. The differences in mass between the various tagged analytes are measured using a mass spectrometer. In this way differential levels of the analyte can be determined. By adding another labeled internal standard, absolute concentration of the species in the medium can be measured.

Analysis Method

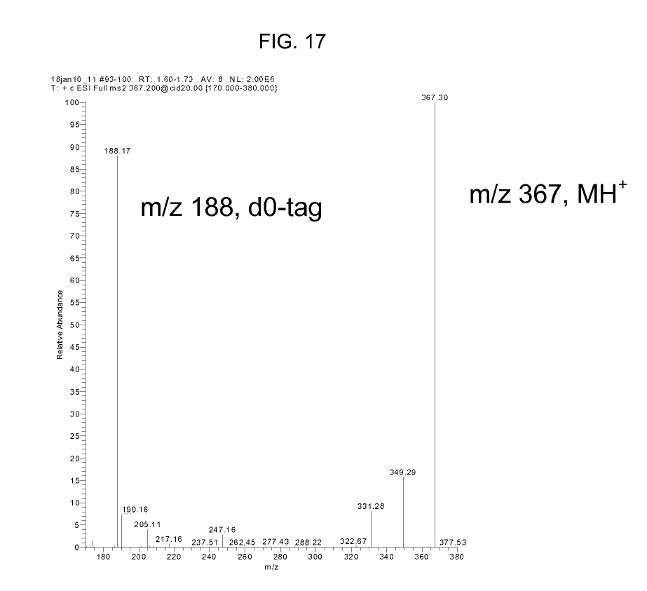

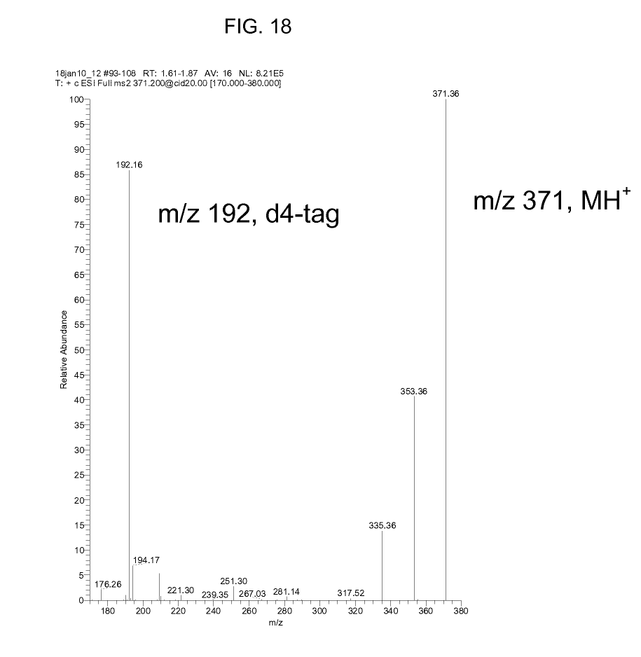

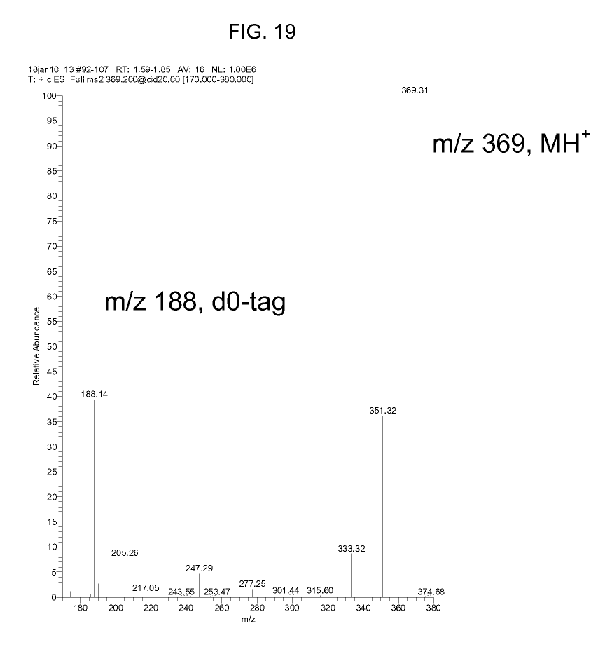

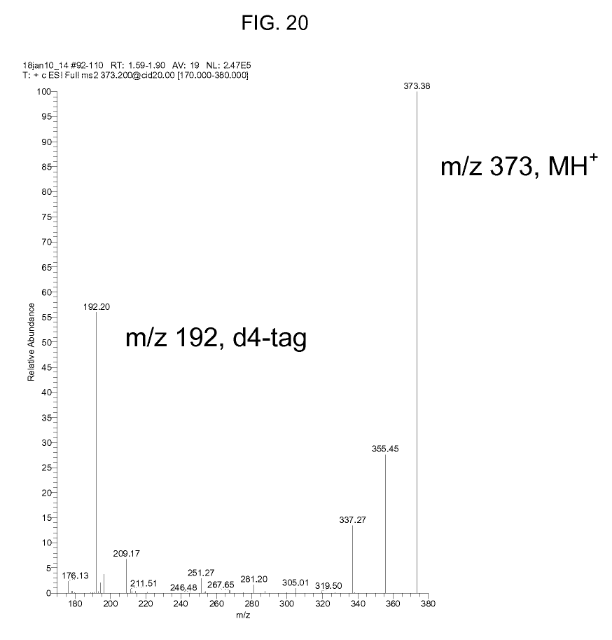

In this method a chemical tag, PPEA (prepared by the methods analogous to those described for Example 1 above) was synthesized with varying numbers of deuterium atoms (0, 4, 8 . . . ). These compounds were then used to prepare derivatives of an analyte, in this case D-glucose. The PPEA tags reacted with D-glucose to form a Schiff base (Scheme 3). The mass shifts between the different species were then monitored by mass spectrometry. Liquid chromatography was used for separation of components followed by electrospray ionization using a triple quadrupole mass spectrometer operating in ms/ms mode. Parent ions of the various combinations were further fragmented by Collision Induced Dissociation (CID) and the product ions were observed, as shown in Table 1. The product ion mass spectra are shown in FIG. 17-20.

Simultaneously, to measure the enrichment of [6,6-²H₂]glucose in a sample, the ratio of the parent ions are measured, e.g. m/z 369/367 using a d0-tag or m/z 373/371 using a d4-tag. The relative concentration of each group in a mixture is monitored using the product ions, e.g. m/z 192 vs 188 for d4- and d0-tags respectively. Again, by using a tagged glucose internal standard (e.g. m/z 196), the absolute concentrations of the unknown glucose samples are also determined in the same run.

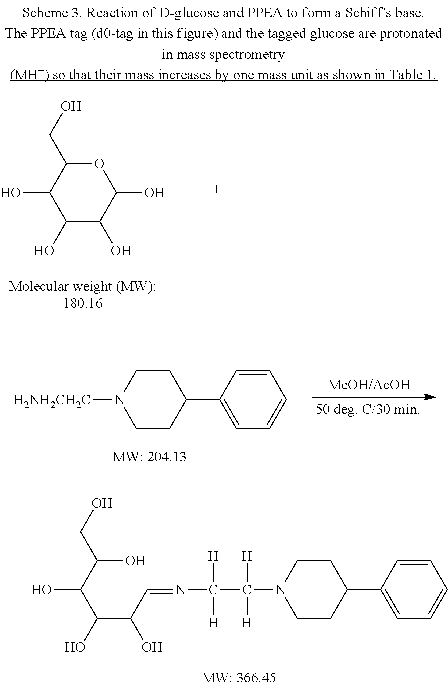

TABLE 1
Parent and product ions produced by various combinations of
labeled forms of glucose and PPEA

| | MH⁺ | product ion (tag) |
|---|---|---|
| PPEA d0-tag | 205 | |
| D0-glucose + d0-tag | 367 | 188 |
| D0-glucose + d4-tag | 371 | 192 |
| D2-glucose + d0-tag | 369 | 188 |
| D2-glucose + d4-tag | 373 | 192 |

A number of embodiments of the invention have been described. Nevertheless, it will be understood that various modifications may be made without departing from the spirit and scope of the invention. Accordingly, other embodiments are within the scope of the following claims.

### Example 3

### Synthesis and Analysis of Isotopically-Labeled Chemical Tages

Amino type isotopically-labeled multiplex chemical tags (iLAQ) were synthesized for the simultaneous quantitation of multiple glucose samples.

The tag was synthesized as a 2-(4-phenylpiperidin-yl-1)ethanamine (PPEA). Two isotopomers of PPEA have been synthesized: M+0 (unlabeled, d0-tag) and M+4 (the 4 methylene hydrogen atoms are replaced by deuterium atoms, d4-tag).

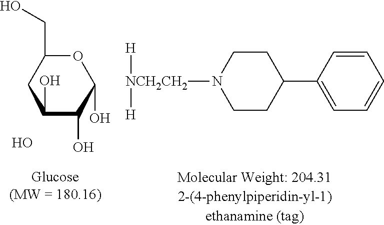

Glucose was derivatized with d0-tag and d4-tag separately to ‘tag’ the glucose, both in a ratio of tag to glucose of 1:1 by weight. The derivatives produced, d0-tag-glucose and d4-tag-glucose, have m/z values of 367 and 371, respectively, in Q1. In the collision cell, the derivative complex was broken to produce the fragment of the tag, 188 and 192, respectively. These fragments were quantified in Q2 to represent the amount of glucose injected because each glucose molecule produces one fragment (1:1 ratio). Therefore, by quantifying d0 and d4 tags, the amounts of glucose in each sample was differentiated (by mass) and quantified (by peak intensity of the fragments).

The two tag-glucose derivatives were analyzed by LC-MS/MS. The results in Table 2 show that the d0-tag-glucose and d4-tag-glucose derivatives, when analyzed separately, have a glucose-to-tag ratio of 1:1, as expected. Then, the d0-tag-glucose and d4-tag-glucose derivatives were mixed, injected (8 pg/injection, other injection amounts were not tested) and quantified simultaneously. The results demonstrate that the ratios for both derivatives remain unchanged. Therefore, the two different samples of the same analyte can be quantified in the same operations, thereby doubling the productivity. The results also indicate a linear dynamic range of the analysis in that the different amounts of injection (100, 20 and 8 pg/injection) did not affect the resulting ratios. This demonstrates it tolerates variability in the amount of analytes injected which is usually the case in routine analyses.

TABLE 2
Simultaneous quantitation of multiple glucose samples

| | | Injected | Glucose/d0- | Glucose/d4- |
|---|---|---|---|---|
| Vial # | Sample name | glucose (pg) | tag ratio | tag ratio |
| 1 | d0-tag glucose | 100 | 1.08 | |
| 2 | d4-tag glucose | 100 | | 0.97 |
| 3 | d0-tag glucose | 100 | 1.05 | |
| 4 | d4-tag glucose | 100 | | 1.00 |
| 5 | d0-tag glucose | 100 | 1.06 | |
| 6 | d4-tag glucose | 100 | | 0.94 |
| 1 | d0-tag glucose | 20 | 1.09 | |
| 2 | d4-tag glucose | 20 | | 1.00 |
| 3 | d0-tag glucose | 20 | 1.05 | |
| 4 | d4-tag glucose | 20 | | 0.94 |
| 5 | d0-tag glucose | 20 | 1.08 | |
| 6 | d4-tag glucose | 20 | | 0.99 |
| 1 | d0-tag glucose | 8 | 1.17 | |
| 2 | d4-tag glucose | 8 | | 0.88 |
| 3 | d0-tag glucose | 8 | 1.08 | |
| 4 | d4-tag glucose | 8 | | 0.93 |
| 5 | d0-tag glucose | 8 | 1.15 | |
| 6 | d4-tag glucose | 8 | | 0.89 |
| 1 + 2 | Vial 1&2 mix | 8 | 1.10 | 0.94 |
| 3 + 4 | Vial 3&4 mix | 8 | 1.09 | 1.08 |
| 5 + 6 | Vial 5&6 mix | 8 | 1.07 | 0.99 |

Comparison on quantitation of two glucose samples separately and mixed

| | | | | |
|---|---|---|---|---|
| separately | d0-tag glucose | 8 | | |
| | Average (n = 3) | | 1.13 | |
| | SEM | | 0.03 | |
| | d4-tag glucose | 8 | | |
| | Average (n = 3) | | | 0.90 |
| | SEM | | | 0.01 |
| mixed | d0-tag glucose | 8 | | |
| | Average (n = 3) | | 1.09 | |
| | SEM | | 0.01 | |
| | d4-tag glucose | 8 | | |
| | Average (n = 3) | | | 1.00 |
| | SEM | | | 0.04 |
| P value | | | 0.2 | 0.09 |

P value: from comparison of ratios obtained separately and simultaneously.

1. A plurality of compounds of Formula I: 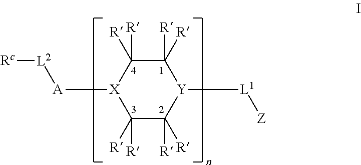
or salts thereof; comprising a non-isobaric series of compounds; wherein:
each compound in the series has the same structural formula but a different isotopic substitution pattern;
n is an integer selected from 0, 1, 2 or 3;
Z is selected from —C(═O)OR¹, —C(═O)X¹, and —NHR²;
X¹ is halogen;
R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein the C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy;
X is selected from C(R′) and ¹³C(R′); and Y is selected from C(R′) and ¹³C(R′); or
X is selected from N and ¹⁵N; and Y is selected from N and 15N;
when n is 1, 2, or 3 and X is N or ¹⁵N, then A is absent; or
when (1) X is C(R′) or ¹³C(R′) and n is 1, 2, or 3; or (2) n is 0, then A is N(R^{f}), or ¹⁵N(R^{f});
L¹ is -G¹-G²-G³-G⁴-G⁵-G⁶-; wherein G¹ is attached to Z;
L² is -E¹-E²-E³-E⁴-E⁵-E⁶-E⁷-E⁸; wherein E¹ is attached to A;
R^{f} is —F¹—F²—F³—F⁴—F⁵—F⁶—F⁷—F⁸—R^{c};
G¹ is —C(R^{a})₂— or —¹³C(R^{a})₂—;
G², G³, G⁴, G⁵, and G⁶ are each independently absent, —C(R^{a})₂—, or —¹³C(R^{a})₂—;
E¹, E², E³, E⁴, E⁵, E⁶, E⁷, and E⁸ are each independently absent, —C(R^{b})₂—, or —¹³C(R^{b})₂—;
F¹, F², F³, F⁴, F⁵, F⁶, F⁷, and F⁸ are each independently absent, —C(R^{b′})₂—, or —¹³C(R^{b′})₂—;
each R^{c} is independently selected from H and D;
each R^{a}, R^{b}, and R^{b′} is independently selected from H and D;
each R′ is independently selected from H and D; and
C1, C2, C3, and C4 of the ring are independently carbon or carbon-13.

2. The plurality of claim 1, wherein said plurality is a plurality of compounds of Formula II: 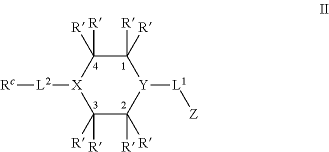
or salts thereof; wherein:
X is selected from N and ¹⁵N;
Y is selected from N and ¹⁵N.

3. The plurality of claim 2, wherein Z is —C(═O)OH.

4. The plurality of claim 2, wherein Z is —NH₂.

5. The plurality of claim 2, wherein L² is absent.

6. The plurality of claim 2, wherein:
L² is selected from -E¹-, E¹-E²-, -E¹-E²-E³-, -E¹-E²-E³-E⁴-, -E¹-E²-E³-E⁴-E⁵-, and -E¹-E²-E³-E⁴-E⁵-E⁶-; and
E¹, E², E³, E⁴, E⁵, and E⁶ are each independently —C(R^{b})₂— or -¹³C(R^{b})₂—.

7. The plurality of claim 2, wherein:
L² is -E¹-E²-; and
E¹ and E² are each independently —C(R^{b})₂— or —¹³C(R^{b})₂—.

8. The plurality of claim 2, wherein:
L² is -E¹-E²-E³-; and
E¹, E², and E³ are each independently —C(R^{b})₂— or —¹³C(R^{b})₂—.

9. The plurality of claim 2, wherein:
L² is -E¹-E²-E³-E⁴-E⁵-E⁶-; and
E¹, E², E³, E⁴, E⁵, and E⁶ are each independently —C(R^{b})₂— or —¹³C(R^{b})₂—.

10. The plurality of claim 2, wherein L¹ is -G¹-.

11. The plurality of claim 2, wherein L¹ is -G¹-G².

12-15. (canceled)

16. The plurality of claim 2, wherein, for the compound with the highest formula weight in the plurality:
C1, C2, C3, and C4 of the ring are each carbon-13;
X is ¹⁵N;
Y is ¹⁵N;
G¹ is —¹³CD₂-;
each R′ is D; and
E¹, E², E³, E⁴, E⁵, and E⁶ are each —¹³CD₂-.

17-21. (canceled)

22. The plurality of claim 1, wherein said plurality is a plurality of compounds of Formula III: 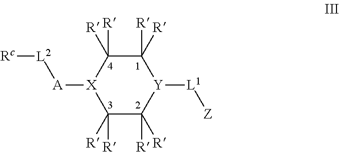
or salts thereof; wherein:
A is —N(R^{f})—, or —¹⁵N(R^{f})—;
X is selected from C(R′) and ¹³C(R′); and
Y is selected from C(R′) and ¹³C(R′).

23-32. (canceled)

33. The plurality of claim 1, wherein said plurality is a plurality of compounds of Formula IV: 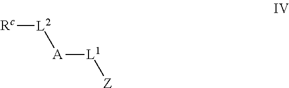
or salts thereof; wherein A is —N(R^{f})—, or —¹⁵N(R^{f})—.

34-37. (canceled)

38. The plurality of claim 33, wherein, for the compound with the highest formula weight in the plurality:
A is —¹⁵N(R^{f})—;
E¹, E², and E³ are each —¹³CD₂-;
F¹, F², and F³ are each —¹³CD₂-; and
G¹ and G² are each —¹³CH₂—.

39-42. (canceled)

43. The plurality of claim 1, wherein one compound is not labeled with carbon-13, nitrogen-15 or D.

44. The plurality of claim 1, comprising at least one compound labeled with carbon-13, nitrogen-15 or both as in any of the preceding claims and further having w D atoms wherein w is 1+(the total number of atoms labeled with carbon-13 or nitrogen-15 in the highest formula weight compound of the plurality which is not labeled with D).

45. The plurality of claim 1, comprising at least one compound labeled with carbon-13, nitrogen-15 or both as in any of the preceding claims, wherein each R′ is D.

46. A kit comprising a plurality of compounds selected from the plurality of claim 1.

47. A method of forming a plurality of labeled analyte samples, comprising reacting independently each of q individual samples of the analyte with a different compound selected from the plurality of compounds of any one of claim 1 to form a plurality of q labeled samples; wherein:
q is equal to the total number of compounds in the plurality of compounds;
each individual sample of the analyte is reacted with a different compound in the plurality of compounds; and
each of said q labeled samples comprises a labeled analyte.

48-51. (canceled)

52. A method of quantifying an analyte, comprising:
mixing together the plurality of q labeled samples of claim 47; and
isolating the labeled analytes; and
quantifying the analyte by conducting an mass spectrometry analysis on the labeled analytes obtained from said isolating;

53-54. (canceled)

55. A compound of Formula Ia: 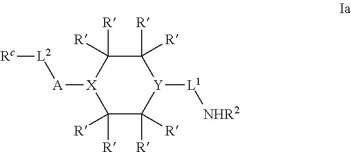
or salt thereof; wherein:
each compound in said plurality has the same structural formula provided that each compound has a different isotopic substitution pattern;
R² is independently selected from H, C₁₋₆ alkyl, C₁₋₆ alkenyl, C₁₋₆ alkynyl, C₁₋₆ haloalkyl, C₆₋₁₀ aryl, C₆₋₁₉ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein said C₆₋₁₀ aryl, C₆₋₁₉ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy;
X is selected from C(R′) and ¹³C(R′); and Y is selected from C(R′) and ¹³C(R′); or
X is selected from N and ¹⁵N; and Y is selected from N and ¹⁵N;
when X is N or ¹⁵N, then A is absent; or
when X is C(R′) or ¹³C(R′), then A is N(R^{f}), or ¹⁵N(R^{f});
L¹ is -G¹-G²-G³-G⁴-G⁵-G⁶-; wherein G¹ is attached to Z;
L² is -E¹-E²-E³-E⁴-E⁵-E⁶-E⁷-E⁸; wherein E¹ is attached to A;
R^{f} is —F¹—F²—F³—F⁴—F⁵—F⁶—F⁷—F⁸—R^{c};
G¹ is —C(R^{a})₂— or —¹³C(R^{a})₂—;
G², G³, G⁴, G⁵, and G⁶ are each independently absent, —C(R^{a})₂—, or —¹³C(R^{a})₂—;
E¹, E², E³, E⁴, E⁵, E⁶, E⁷, and E⁸ are each independently absent, —C(R^{b})₂—, or —¹³C(R^{b})₂—;
F¹, F², F³, F⁴, F⁵, F⁶, F⁷, and F⁸ are each independently absent, —C(R^{b′})₂—, or —¹³C(R^{b′})₂—;
each R^{c} is independently selected from H and D;
each R^{a}, R^{b}, and R^{b′} is independently selected from H and D;
each R′ is independently selected from H and D; and
C1, C2, C3, and C4 of the ring are independently carbon or carbon-13;
provided that the compound contains at least one atom selected from ¹³C, ¹⁵N, or D.

56. A compound of claim 55, wherein R² is H.

57. A compound of Formula V: 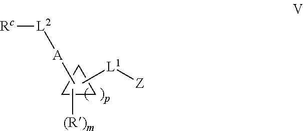
or salt thereof; wherein:
Z is selected from —C(═O)OR¹, —C(═O)X¹, and —NHR²;
A is —N(R^{f})— or —¹⁵N(R^{f})—;
L¹ is -G¹-G²-G³-G⁴-G⁵-G⁶-; wherein G¹ is attached to Z;
L² is -E¹-E²-E³-E⁴-E⁵-E⁶-E⁷-E⁸; wherein E¹ is attached to A;
R^{f} is —F¹—F²—F³—F⁴—F⁵—F⁶—F⁷—F⁸—R^{c};
G¹ is —C(R^{a})₂— or —¹³C(R^{a})₂—;
G², G³, G⁴, G⁵, and G⁶ are each independently absent, —C(R^{a})₂—, or —¹³C(R^{a})₂—;
E¹, E², E³, E⁴, E⁵, E⁶, E⁷, and E⁸ are each independently absent, —C(R^{b})₂—, or —¹³C(R^{b})₂—;
F¹, F², F³, F⁴, F⁵, F⁶, F⁷, and F⁸ are each independently absent, —C(R^{b′})₂—, or —¹³C(R^{b′})₂—;
p is a integer selected from 1, 2, 3, 4, and 5;
m is integer equal to 2+(2*p);
the carbon atoms of the ring in Formula V are each independently carbon or carbon-13;
each R^{c} is independently selected from H and D;
each R^{a}, R^{b}, and R^{b′} is independently selected from H and D;
each R′ is independently selected from H and D;
X¹ is halogen; and
R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₆ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein said C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy.
provided that the compound contains at least one atom selected from ¹³C, ¹⁵N, or D.

58. The compound of claim 57, wherein said compound has Formula Va: 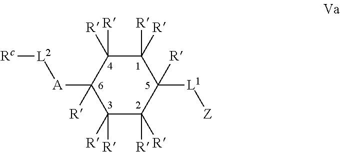
or salt thereof; wherein:
Z is selected from —C(═O)OR¹, —C(═O)X¹, and —NHR²;
each R′ is independently selected from H and D;
A is —N(R^{f})— or —¹⁵N(R^{f})—;
L¹ is -G¹-G²-G³-G⁴-G⁵-G⁶-; wherein G¹ is attached to Z;
L² is -E¹-E²-E³-E⁴-E⁵-E⁶-E⁷-E⁸-; wherein E¹ is attached to A;
R^{f} is —F¹—F²—F³—F⁴—F⁵—F⁶—F⁷—F⁸—R^{b};
G¹ is —C(R^{a})₂— or —¹³C(R^{a})₂—;
G², G³, G⁴, G⁵, and G⁶ are each independently absent, —C(R^{a})₂—, or —¹³C(R^{a})₂—;
E¹, E², E³, E⁴, E⁵, E⁶, E⁷, and E⁸ are each independently absent, —C(R^{b})₂—, or —¹³C(R^{b})₂—;
F¹, F², F³, F⁴, F⁵, F⁶, F⁷, and F⁸ are each independently absent, —C(R^{b′})₂—, or ¹³C(R^{b′})₂—;
each R^{c} is independently selected from H and D;
each R^{a}, R^{b}, and R^{b′} is independently selected from H and D;
each R′ is independently selected from H and D;
X¹ is halogen; and
R¹ and R² are each independently selected from H, C₁₋₆ alkyl, C₁₋₆ alkenyl, C₁₋₆ alkynyl, C₁₋₆ haloalkyl, C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl; wherein said C₆₋₁₀ aryl, C₆₋₁₀ aryl-C₁₋₃ alkyl, C₁₋₉ heteroaryl, C₁₋₉ heteroaryl-C₁₋₃ alkyl, C₃₋₇ cycloalkyl, C₂₋₉ heterocycloalkyl, C₃₋₇ cycloalkyl-C₁₋₃ alkyl, and C₂₋₉ heterocycloalkyl-C₁₋₃ alkyl are each optionally substituted by 1, 2, 3, or 4 groups independently selected from halogen, cyano, nitro, C₁₋₄ alkyl, C₁₋₄ haloalkyl, C₁₋₄ alkoxy, and C₁₋₄ haloalkoxy;
C1, C2, C3, C4, C5, and C6 of the ring in Formula Va are independently carbon or carbon-13;
provided that the compound contains at least one atom selected from ¹³C, ¹⁵N, or D.

59-60. (canceled)
